(19) United States
(12) Patent Application Publication
(10) Pub. No.: US 2010/0285579 A1
(43) Pub. Date: Nov. 11, 2010

(54) PREANTRAL FOLLICLE DERIVED EMBRYONIC STEM CELLS

(75) Inventors: Jeong Mook Lim, Seoul (KR); Jae Yong Han, Seoul (KR); Hee Bal Kim, Seoul (KR); Seung Tae Lee, Gwangju (KR); Jong Eun Ihm, Seoul (KR)

Correspondence Address: THE NATH LAW GROUP, 112 South West Street, Alexandria, VA, 22314, US

(73) Assignee: SEOUL NATIONAL UNIVERSITY INDUSTRY FOUNDATION, Seoul (KR)

(21) Appl. No.: 12/301,465

(22) PCT Filed: May 19, 2006
(86) PCT No.: PCT/KR2006/001891

§ 371 Date: Mar. 15, 2010

(51) Int. Cl. C12N 5/075; C12N 5/02; C12N 5/00

(52) U.S. Cl. 435/353; 435/377; 435/366; 435/354; 435/352; 435/325

(57) ABSTRACT

The present invention relates to a method for producing a preantral follicle-derived embryonic stem cell and a preantral follicle-derived embryonic stem cell. The present method comprises the steps of (a) obtaining a preantral follicle from mammalian ovaries; (b) growing the preantral follicle in vitro; (c) maturing an oocyte in vitro present in the cultured preantral follicle; (d) activating the matured oocyte for parthenogenesis; (e) culturing the activated oocyte to form a blastocyst; and (f) culturing inner cell mass (ICM) cells of the blastocyst to produce the preantral follicle-derived embryonic stem cell.

### BACKGROUND OF THE INVENTION

1. Field of the Invention

The present invention relates to a method for producing a preantral follicle-derived embryonic stem cell and a preantral follicle-derived embryonic stem cell.

2. Description of the Related Art

There exist numerous preantral (primordial, primary and secondary) follicles in the ovaries, but in one's life only less than 1% of the follicles typically develop into the Graafian follicles that could release mature oocytes into the fertilization site [1]. The rest remain “developmentally dormant” in ovarian tissue and finally became degenerated via apoptosis. In the field of animal biotechnology efforts have been made over the last decade to utilize preantral follicles for increasing reproductivity. As results, follicular oocytes derived from in vitro-cultured secondary follicles have been developed into blastocysts following IVF and culture, and full-term development of embryos after transfer has been achieved in F1 mouse of C57BL6×CBA [2, 3]. On the other hand, a marvelous success to generate preimplantation embryos by in vitro manipulation of embryonic stem (ES) cells has recently been reported [4]. Further application of the preantral follicle culture has been subsequently suggested for developing novel medical technology.

Nevertheless, basic information on preantral follicle culture has not been reported yet and a standard protocol of follicle manipulation has not been established. Furthermore, the feasibility of the immature follicle culture technique should be confirmed in other strains and species and the development of the standard method is definitely necessary for both preclinical model researches and clinical application of novel biotechnologies.

Recruitment of immature follicles to obtain large quantities of developmentally competent oocytes has been considered for developing novel medical biotechnologies as well as for improving the reproductive performance of domestic animals. Eppig and colleagues [2; 21] firstly succeeded in producing live births after in-vitro fertilization of oocytes derived from in-vitro-cultured late secondary follicles. Several attempts have been made to optimize the culture protocol of preantral follicles; for example, microbead and three-dimensional culture systems have recently been tested [5; 22; 23]. In addition, non-human primate ES cells were derived after parthenogenetic activation of in-vivo-matured oocytes [24], and efforts to develop a cryopreservation system for preantral follicle have also been made [25]. However, previous attempts to establish ES cells from in-vitro-cultured preantral follicles were unsuccessful.

Throughout this application, various publications and patents are referenced and citations are provided in parentheses. The disclosures of these publications and patents in their entities are hereby incorporated by references into this application in order to more fully describe this invention and the state of the art to which this invention pertains.

### SUMMARY OF THE INVENTION

Under such circumstances, the present inventors have made intensive researches to meet long-felt need in the art, and as a result, developed a novel method for successfully securing preantral follicles as an alternative source of embryonic stem cells.

Accordingly, it is an object of this invention to provide a method for producing a preantral follicle-derived embryonic stem cell.

It is another object of this invention to provide a preantral follicle-derived embryonic stem cell.

Other objects and advantages of the present invention will become apparent from the detailed description to follow and together with the appended claims and drawings.

### BRIEF DESCRIPTION OF THE DRAWINGS

FIG. 1 represents the classification of preantral follicles at retrieval (×600). The primary follicle (A) consisted of single layer of granulosa cell and basement membrane, while the early (B) and late (C) secondary follicles had multiple layers of granulosa cells. The classification of early and late secondary follicle was determined by their size (Scale bar; 50 μm).

FIG. 2 shows the morphology of preantral follicles at retrieval (×120). The preantral follicles were collected either singly (A) or in group (B). The follicles collected in group were difficult to separate from each other and were not suitable for single preantral culture using microdroplet (250 μm; scale bar).

FIG. 3 represents the morphological difference of preantral follicles and follicular oocytes retrieved from mouse (C57BL6/DBA2) ovaries by different methods. Either a mechanical method using syringe needle or an enzymatic method using collagenase and DNAase was employed. (A) The follicle retrieved by the mechanical method (day 0 of culture): basement membrane was intact and several theca cells still attached with the membrane (×600). (B) The follicle retrieved by the enzymatic method (day 0 of culture): the basement membrane was not visible and the theca cells were completely detached from the membrane (×600). (scale bar; 50 μm)

FIG. 4 represents the development of the preantral follicles retrieved from mouse (C57BL/DBA) ovaries during in vitro culture. Primary follicles retrieved by a mechanical method were cultured in α-MEM-glutamax medium supplemented with fetal bovine serum, insulin, transferrin, selenium, FSH and antibiotics. (A) Follicular stage: the preantral follicle remained spherical shape and distinct basement membrane was visible (×600). (B) Diffuse stage: the granulosa cells that enclosed oocyte proliferated and outgrew (×600). (C) Pseudoantral stage: the follicle formed antrum-like translucent structure by the proliferation of granulose cells (×300). (D) Degenerative stage: the granulose cells became degenerated after the oocyte spontaneously dispatched from granulosa cell complex (×300). (50 μm scale bar in A and B and 100 μm in C and D).

FIGS. 5A-5F represent in vitro-growth of preantral follicles retrieved by different methods. Primary, early secondary and late secondary follicles were cultured in α-MEM-glutamax medium supplemented with fetal bovine serum, insulin, transferrin, selenium, FSH and antibiotics, and in vitro-growth to reach the follicle (black bar), diffuse (white), pseudoantral (diagonal) and degenerative (hatched) stages was monitored daily under an inverted microscope. The values indicated the mean percentage±SD. (A and B) Growth of primary follicles: more follicles retrieved by a mechanical method developed into the pseudoantral stage on day 11 and 12 of culture, while all follicles retrieved by an enzymatic method ceased their development at the diffuse stage until day 4 of culture. (C and D) Growth of early secondary follicle: the incidence of pseudoantral stage was peaked on day 10 (74%) and on day 9 (70%) of culture in the mechanical and the enzymatic method, respectively. (E and F) Growth of late secondary follicles: the peak of pseudoantrum formation was on day 7 (73%) and day 6 (80%) of culture in the mechanical and the enzymatic method, respectively. Different letters in the same stage of follicle development demonstrated a significant (P<0.05) difference among observation times.

FIGS. 6A-6E represent the meiotic maturation of oocytes derived from the psedoantral stage of primary, early secondary or late secondary follicles retrieved by different methods. Maturational status was monitored daily and hCG and epidermal growth factor was added into culture medium 16 hours prior to the culture for oocyte maturation. The values indicated the mean percentage±SD and the percentage of oocytes developing to germinal vesicle (GV), germinal vesicle breakdown (GVBD) and metaphase II (MII) stages were monitored at each time of observation. (A) Maturation of oocytes grown in primary follicles retrieved by a mechanical method. MII stage oocytes were detected between 10 to 13 days (13 to 27%). Maturation of oocytes grown in early secondary follicles retrieved by the mechanical (B) and the enzymatic (C) method. Significant increase in MII oocytes was detected on day 9 (47%) in the mechanical and on day 7 (54%) in the enzymatic method. Maturation of oocytes grown in late secondary follicles retrieved by the mechanical (D) and the enzymatic (E) method. Significant increase in MII oocytes was detected from day 5 to day 7 (28% and 78%) in the mechanical and the enzymatic method, respectively. Different letters in the same category of follicle development demonstrated a significant (P<0.05) difference among observation times.

FIG. 7 represents the morphological difference of follicular oocytes derived from preantral follicles isolated by an enzyme treatment. (A) Oocytes grown in the follicle retrieved by the mechanical method (day 11 of culture): First polar body was visible and thick zona pellucida and narrow perivitelline space was observed (×600). (B) Oocytes grown in the follicle retrieved by the enzymatic method: First polar body was visible, but thin zona pellucida and wide perivitelline space was detected (×600). (C) Oocytes ovulated in vivo (scale bar; 50 μm).

FIG. 8 represents the development of preantral follicles retrieved from the ovaries of F1 (C57BL6×DBA2) mice during in vitro culture. Mechanically retrieved secondary follicles were cultured in MEM-glutamax medium supplemented with fetal bovine serum, insulin, transferrin, selenium, FSH and antibiotics. (A) Follicular stage: the follicle remained spherical during culture and a distinct basement membrane is visible (×600). (B) Diffuse stage: granulosa cells that enclose the oocyte have proliferated and grown out (×600). (C) Pseudoantral stage: the follicle has formed an antrum-like translucent structure due to the proliferation and differentiation of granulosa cells (×300). (D) Degenerative stage: the granulosa cells have degenerated after the oocyte spontaneously detached from the granulosa cell complex (×300). (50 μm scale bar in A, B; 100 μm in C and D).

FIG. 9 represents the characterization of follicle-derived, homozygous embryonic stem (ES) cells (A) established by parthenogenetic activation and the subsequent subculture of inner cell mass (ICM) cell colonies in modified knock-out DMEM supplemented with a 3:1 mixture of fetal bovine serum and knock-out serum replacement. Follicle-derived mouse ES cells were characterized using seven stem cell-specific markers: alkaline phosphatase (AP; F) and anti-stage specific embryonic antigen (SSEA)-1 (B), anti-SSEA-3 (C), anti-SSEA-4 (D), Oct-4 (E), anti-integrin α6 (G), and anti-integrin β1 (H) antibodies. The established ES cells stained positively with all the specific markers, except with anti-SSEA-3 and anti-SSEA-3 antibodies, which share identity with the mouse ES cells of other origins. Scale bar, 50 μm.

FIG. 10 represents in vitro differentiation of follicle-derived, homozygous embryonic stem (ES) cells (A) established by parthenogenetic activation and subsequent subculture of inner cell mass (ICM) cell colonies in modified knock-out DMEM supplemented with the 3:1 mixture of fetal bovine serum and knock-out serum replacement. The colonies of follicle-derived ES cells were cultured in leukemia inhibitory factor-free medium for spontaneous differentiation into embryoid bodies and immunocytochemistry of the embryoid bodies was conducted using three germ layer specific markers of Neural cadherin adhesion molecule (NCAM for ectoderm, A), muscle actin (B; mesoderm), α-feto protein (C; endoderm), S-100 (D; ectoderm), Desmin (E; mesoderm) and Troma-1 (F; endoderm). The cells consisting of embryoid bodies were positively stained with one of the markers tested. Scale bar indicates 50 μm.

FIG. 11 demonstrates the neuronal differentiation of preantral follicle-derived homozygous embryonic stem (ES) cells. (A, E) Phase contrast images of differentiated follicle-derived, autologous ES cells in modified N2B27 medium. Tuj1-positive (B) and Nestin-positive (C) neurons generated 7-10 days after replating on fibronectin. (D) Merged image of Tuj1-positive (B) and Nestin-positive (C) neurons. GFAP-positive astrocytes (F) and O4-positive oligodendrocytes (G) generated 11-14 days after replating on fibronectin, respectively. (H) Merged image of GFAP-positive astrocytes and O4-positive oligodendrocytes. Scale bar=40 μm.

FIG. 12 represents the teratoma formation of follicle-derived, homozygous embryonic stem (ES) cells (A) established by parthenogenetic activation and subsequent subculture of inner cell mass (ICM) cell colonies in modified knock-out DMEM supplemented with the 3:1 mixture of fetal bovine serum and knock-out serum replacement 8 weeks after transplantation into NOD-SCID mouse. The morphology of the teratoma was examined by staining of paraffin enblock with hematoxylin and eosin. The morphology of the teratoma was examined by staining of paraffin enblock with hematoxylin and eosin. The teratoma contains glandular stomach-like structure (A), exocrine pancreatic tissue (B) and respiratory epithelium with cilia (arrow head; C) of endodermal cells, stratified squamous epithelium with keratin (D), neuroepithelial rosette (E), pigmented retinal epithelium (F) and sebaceous gland (G) of ectodermal cells, and adipocytes (arrow head; H) and skeletal muscle bundles (arrow; H) of mesodermal cells. Scale bars=200 μm.

### DETAILED DESCRIPTION OF THIS INVENTION

In one aspect of this invention, there is provided a method for producing a preantral follicle-derived embryonic stem cell, which comprises the steps of: (a) obtaining a preantral follicle from mammalian ovaries; (b) growing the preantral follicle in vitro; (c) maturing an oocyte in vitro present in the cultured preantral follicle; (d) activating the matured oocyte for parthenogenesis; (e) culturing the activated oocyte to form a blastocyst; and (f) culturing inner cell mass (ICM) cells of the blastocyst to produce the preantral follicle-derived embryonic stem cell.

### Preparation of Preantral Follicles

The most striking feature of the present invention is to use preantral follicles as a source for producing embryonic stem (ES) cells. To our best knowledge, it has not been reported yet that preantral follicles can be successfully employed to establish embryonic stem cell lines.

The term “preantral follicles” used herein refers to the follicles that did not form antral cavity (antrum), which comprises more than one layer of granulosa cells and immature oocytes arrested before the metaphase II stage. The term “preantral follicle” includes primordial, primary and secondary follicle (early, mid and late stage), but tertiary and Grrafiaan follicles that already form fluid-filled antrum are excluded from this category.

The phrase “preantral follicle-derived” used herein in conjunction with ES cells means that ES cells are prepared in vitro from preantral follicles as a starting material. In other words, preantral follicles are grown, maturated and activated in vitro for providing ES cells.

Preantral follicles are isolated from ovaries in accordance with various methods known to one skilled in the art. For example, preantral follicles may be retrieved mechanically using a suitable device, e.g., needle [5]. Otherwise, an enzymatic retrieval method using suitable proteinases (e.g., collagenase and trypsin) and/or DNAase may be employed for the isolation of preantral follicles. According to a preferred embodiment, the proteinase is collagenase type I and DNAase I.

According to a preferred embodiment, the isolation of preantral follicles is conducted by the mechanical method, more preferably using a needle, most preferably a 10-40 gauge needle. The term “mechanical method” used herein with reference to the isolation of preantral follicles refers to methods for directly retrieving preantral follicles by use of devices to mechanically isolate preantral follicles form ovaries. The mechanical isolation method is advantageous over an enzymatic method in the senses that it allows for obtaining larger number of follicles than the enzymatic method and further shows increased viability of oocytes obtained from preantral follicles with the comparison to the enzymatic method. The enzymatic retrieval method is very likely to damage basement membrane of preantral follicles, finally resulting in the decrease in the efficiency of ES cell production.

A population of preantral follicles isolated comprises generally primary follicle, early secondary follicle and late secondary follicle.

The preantral follicles may be obtained from mammals, preferably, humans, bovines, sheep, ovines, pigs, horses, rabbits, goats, mice, hamsters and rats, more preferably, humans, mice and rats and most preferably, mice.

### In Vitro Growth of Preantral Follicles

Preantral follicles isolated are then cultured in a medium to reach a suitable growth stage.

According to a preferred embodiment, the preantral follicle used in the step is an early secondary follicle. The early secondary follicle may be selected on the basis of size and morphological criteria: 100 to 125 μm in diameter, and round structure with multiple layers of granulosa cells and a follicular oocyte.

A medium useful in the step includes any conventional medium containing human follicle stimulating hormone (hFSH) and/or luteinizing hormone (LH) for mammalian follicle or oocyte culture in the art. For example, the medium includes Eagles's MEM [Eagle's minimum essential medium, Eagle, H. Science 130:432(1959)], α-MEM [Stanner, C. P. et al., Nat. New Biol. 230:52(1971)], Iscove's MEM [Iscove, N. et al., J. Exp. Med, 147:923(1978)], 199 medium [Morgan et al., Proc, Soc. Exp. Bio. Med., 73:1(1950)], CMRL 1066, RPMI 1640 [Moore et al., J. Amer. Med. Assoc. 199:519(1967)], F12 [Ham, Proc. Natl. Acad. Sci. USA 53:288(1965)], F10 [Ham, R. G. Exp. Cell Res. 29:515(1963)], DMEM [Dulbecco's modification of Eagle's medium, Dulbecco, R. et al., Virology 8:396(1959)], a mixture of DMEM and F12 [Barnes, D. et al., Anal. Biochem. 102:255(1980)], Way-mouth's MB752/1 [Waymouth, C. J. Natl. Cancer Inst. 22:1003(1959)], McCoy's 5A [McCoy, T. A., et al., Proc. Soc. Exp, Biol. Med, 100:115(1959)], a series of MCDB [Ham, R. G. et al., In Vitro 14:11(1978)] and their modifications. The detailed description of media is found in R. Ian Freshney, Culture of Animal Cells, A Manual of Basic Technique, Alan R. Liss, Inc., New York, the teaching of which is incorporated herein by reference in its entity.

Preferably, the medium for growing preantral follicle in vitro is α-MEM-glutamax medium, more preferably, supplemented with fetal bovine serum (FBS), insulin, transferrin, selenium, human follicle stimulating hormone (hFSH), luteinizing hormone (LH) and/or antibiotics (such as penicillin and streptomycin). Where primary follicles are used in this step, it is preferred that α-MEM-glutamax medium is free from ribonucleoside and deoxyribonucleoside. More preferably, in the case of using primary follicles, ribonucleoside and deoxyribonucleoside-free α-MEM-glutamax medium containing supplements described above is initially employed and thereafter ribonucleoside/deoxyribonucleoside-containing α-MEM-glutamax medium supplemented with FBS, insulin, transferrin, selenium, hFSH and/or antibiotics is employed after the diameter of the cultured follicles reaches approximately 100 μm. Where early secondary follicles are used in this step, it is preferred that ribonucleoside/deoxyribonucleoside-containing α-MEM-glutamax medium supplemented with FBS, insulin, transferrin, selenium, hFSH, LH and/or antibiotics is employed throughout this step.

It is preferred that the culture for growing preantral follicle in vitro is carried out in accordance with a single cell culture system [3]. More specifically, the culturing is performed by placing singly follicles in culture droplets containing media described hereinabove which is overlaid with mineral oil.

Where early secondary follicles (in particular, derived from mouse) are used, the period of time for in vitro growth of preantral follicles is preferably 6-13 days, more preferably, 8-10 days and most preferably about 9 days.

In general, in vitro-growth of preantral follicles is classified into four stages, namely the follicular, diffuse, pseudoantral and degenerative stages. According to a preferred embodiment, preantral follicles are cultured to reach the pseudoantral stage. The preantral follicles at the pseudoantral stage may be characterized as forming antrum-like, granulosa cell-free area. Maximal expansion of granulosa cells allows for the creation of an empty space between the granulosa cell matrix, and the basement membrane of the follicle is not visible. Intrafollicular oocyte and its adjacent granulosa (cumulus) cells spontaneously are dispatched (released) from the cell complex.

### In Vitro Maturation of Oocytes in Follicles

The preantral follicles entering a suitable growth stage, preferably pseudoantral stage, are then matured in vitro by the treatment of suitable hormones and/or growth factors.

According to a preferred embodiment, human chorionic gonadotrophin (hCG) is used for maturation of follicular oocytes. More preferably, a combination of human chorionic gonadotrophin and epidermal growth factor (EGF) is used to permit follicular oocytes to be matured. The amount of hCG used ranges from 1.0 to 20 IU (International Unit)/ml, preferably, 1.0-20 IU/ml, more preferably, 1.5-10 IU/ml, still more preferably, 2.0-5 IU/ml, and most preferably, 2.0-3.0 IU/ml. The amount of EGF used is in the range of 1.0-20 ng/ml, preferably, 2.0-10 ng/ml, more preferably, 3.0-7.0 ng/ml, and most preferably, about 5 ng/ml.

The oocyte maturation takes 2-30 hr, preferably, 5-25 hr, more preferably, 10-25 hr, and most preferably 16-18 hr.

The preantral follicles entering a suitable growth stage, preferably, pseudoantral stage, are matured to develop to a suitable maturation stage, preferably, the metaphase II stage. Metaphase II refers to a stage of development wherein the DNA content of a cell consists of a haploid number of chromosomes with each chromosome represented by two chromatids.

Oocyte maturation (developed to the metaphase II stage) may be determined by the extrusion of the first polar body and by detecting mucification and expansion of cumulus cells enclosing oocyte.

### Activation of Matured Oocyte for Parthenogenesis

Following the maturation, the oocytes are then activated for parthenogenesis.

According to a preferred embodiment, cumulus cells surrounding a mature oocyte are removed prior to the treatment for parthenogenesis. Preferably, the removal of cumulus cells is carried out by mechanical pipetting in a suitable medium. More preferably, the medium is one containing hyaluronidase as well as NaCl, KCl, CaCl₂, KH₂PO₄, MgSO₄, NaHCO₃, HEPES, sodium lactate, sodium pyruvate, glucose, antibiotics (preferably, penicillin and streptomycin) and/or bovine serum albumin (BSA). Most preferably, the medium is M2 medium.

Parthenogenesis may be carried out in accordance with various methods known to one of skill in the art. For instance, the oocyte activation for parthenogenesis involves exposing oocytes to ethanol, electroporation, calcium ionophore, ionomycine or inositol 1,4,5-triphosphate to increase the intracellular Ca²⁺ ion concentration in oocytes, in combination with treatments that temporarily inhibits protein synthesis or microfilament synthesis. Preferably, SrCl₂ and/or cytochalasin B is used for parthenogenesis of mature oocytes. More preferably, the parthenogenesis is performed in KSOM [Potassium-enriched Simplex Optimized Medium, Lawitts, J. A. and Biggers, J. D., Methods Enzymol., 225:153-164(1993)] medium supplemented with SrCl₂ and/or cytochalasin B. Most preferably, mature oocytes are activated parthenogenetically by culturing in Ca²⁺-free KSOM medium supplemented with SrCl₂ and cytochalasin B.

The content of SrCl₂ for parthenogenesis ranges from 5 to 25 mM, preferably, 5-20 mM, more preferably, 7-15 mM, and most preferably about 10 mM. The content of cytochalasin B for parthenogenesis ranges from 2.5 to 15 μg/ml, preferably, 2.5-10 μg/ml, more preferably, 4-7 μg/ml, and most preferably about 5 μg/ml. The culture for parthenogenesis is performed for 1-20 hr, preferably, 2-15 hr, more preferably, 2-10 hr, and most preferably, 3-5 hr.

The accomplishment in the parthenogenesis of mature oocytes may be evaluated by determining the capacity of matured oocytes to form pronucleus.

### Development of Activated Oocyte to Blastocyst

The parthenogentically activated oocytes are cultured to develop into blastocyst stage.

The medium for developing the activated oocytes into blastocyst may have any of several formulas. For example, suitable medium sources are as follows: Dulbecco's modified Eagle's medium (DMEM), knock DMEM, DMEM containing fetal bovine serum (FBS), DMEM containing serum replacement, Chatot, Ziomek and Bavister (CZB) medium, Ham's F-10 containing fetal calf serum (FCS), Tyrodes-albumin-lactate-pyruvate (TALP), Dulbecco's phosphate buffered saline (PBS), Eagle's and Whitten's media. Preferably, the medium for parthenogentically activated oocytes to be developed to blastocyst is a Chatot, Ziomek and Bavister (CZB) medium. The CZB medium comprises NaCl, KCl, KH₂PO₄, MgSO₄, CaCl₂, NaHCO₃, sodium lactate, sodium pyruvate, glutamine, EDTA and BSA (bovine serum albumin). More preferably, the CZB medium further comprises Hb (preferably, methemoglobin type) and β-mercaptoethanol. The detailed description of media is found in R. Ian Freshney, Culture of Animal Cells, A Manual of Basic Technique, Alan R. Liss, Inc., New York, WO 97/47734 and WO 98/30679, the teachings of which are incorporated herein by reference in their entities.

According to a preferred embodiment, the culture of parthenogentically activated oocytes is carried out in accordance with a single cell culture system [3]. More specifically, the culturing is performed by placing singly oocytes in culture droplets containing media described hereinabove which is overlaid with mineral oil.

The period of time for culture parthenogentically activated oocytes ranges 2-10 days, preferably 2-8 days, more preferably 4-6 days, and most preferably about 5 days.

The development of parthenogenetically activated oocytes to blastocyst stage may be determined by evaluating a typical morphology of embryo consisting of an inner cell mass, a trophoblast and a blastocoele.

### Production of Preantral Follicle-Derived Embryonic Stem Cell

Following the formation of blastocysts, the blastocyst is cultured to produce preantral follicle-derived embryonic stem cells.

Preferably, the blastocysts are freed from zona pellucida and then cultured. After culturing for a suitable period of time, the ICM (inner cell mass)-derived cell colonies are mechanically or enzymatically retrieved and then subcultured for establishing preantral follicle-derived embryonic stem cell lines. Alternatively, ICM separated from blastocysts of step (e) may be used in culturing for producing follicle-derived embryonic stem cells.

A medium useful in this step includes any conventional medium containing LIF (Leukemia inhibition factor) for obtaining mammalian ES cells known in the art. For example, the medium includes Dulbecco's modified Eagle's medium (DMEM), knock DMEM, DMEM containing fetal bovine serum (FBS), DMEM containing serum replacement, Chatot, Ziomek and Bavister (CZB) medium, Ham's F-10 containing fetal calf serum (FCS), Tyrodes-albumin-lactate-pyruvate (TALP), Dulbecco's phosphate buffered saline (PBS), and Eagle's and Whitten's media. Preferably, the culture medium is knock-out Dulbecco's minimal essential medium (KDMEM) containing LIF supplemented with β-mercaptoethanol, nonessential amino acids, L-glutamine, antibiotics (preferably, penicillin and streptomycin) and/or a mixture of FBS and knock-out serum replacement. The detailed description of media is found in R. Ian Freshney, Culture of Animal Cells, A Manual of Basic Technique, Alan R. Liss, Inc., New York, WO 97/47734 and WO 98/30679, the teachings of which are incorporated herein by reference in their entities.

According to a preferred embodiment, the blastocyst or ICM is cultured on a feeder cell layer. Suitable feeder layers include fibroblasts and epithelial cells derived from various animals, for example, mouse embryonic fibroblasts, human fibroblast-like cells, chicken fibroblasts, uterine epithelial cells, STO and SI-m220 feeder cell lines, and BRL cells. A preferable feeder cell is an embryonic fibroblast derived from mammals, advantageously, mouse. Preferably, the feeder cell is mitotically inactive, for example, by treatment with anti-mitotic agent such as mitomycin C, to prevent it from outgrowing the ES cells it is supporting.

The preparation of embryonic stem cells may be evaluated by maker assays using alkaline phosphatase (AP), anti-stage-specific embryonic antigen (SSEA) antibodies such as anti-SSEA-1, anti-SSEA-3 and anti-SSEA-4 antibodies, anti-integrin α6 antibody, and anti-integrin β1 antibody. In addition, the embryonic stem cells finally prepared by the invention may be confirmed by analyzing their potentials to form embryonic body in the absence of LIF and teratoma. Meanwhile, the karyotyping of the embryonic stem cells produced may show that they are originated from preantral follicles.

In another aspect of this invention, there is provided a preantral follicle-derived embryonic stem cell, wherein the embryonic stem cell has the same karyotype as an oocyte present in the preantral follicle, is stainable with alkaline phosphatase (AP), and capable of forming an embryonic body and teratoma.

The preantral follicle-derived embryonic stem cell has the same karyotype as its mother cell, i. e., oocyte in the preantral follicle. In addition, the preantral follicle-derived embryonic stem cell of this invention exhibits some characteristics common to embryonic stem cells, for example, being stainable with alkaline phosphatase (AP) and capable of forming an embryonic body and teratoma.

The term “stainable” used herein with reference to embryonic stem cells means that cells are positively stained with or reactive to cell surface binding ligands such as AP, anti-SSEA antibody, anti-integrin α6 antibody and anti-integrin β1 antibody.

The preantral follicle-derived ES cell of this invention is pluripotent. The term “pluripotent” means that cells has the ability to develop into any cell derived from the three main germ cell layers. When transferred into SCID mice, a successful preantral follicle-derived ES cell will differentiate into cells derived from all three embryonic germ layers. In addition, when cultured in the absence of LIF, the preantral follicle-derived ES cell of this invention forms an embryonic body being positive for markers specific for any of the three germ layers: neural cadherin adhesion molecule and S-100 for the ectodermal layer; muscle actin and desmin for the mesodermal layer; and α-fetoprotein and Troma-1 for endodermal cells.

According to a preferred embodiment, the embryonic stem cell of this invention is derived from an early secondary follicle. The embryonic stem cell of this invention is derived from an early secondary follicle of mammals, preferably, human, bovine, sheep, ovine, pig, horse, rabbit, goat, mouse, hamster or rat. According to an embodiment of this invention, the embryonic stem cell of this invention is derived from an early secondary follicle of rodents such as mouse. Exemplarily, the embryonic stem cell of this invention is FpB6D2-snu-1 under accession No. KCLRF-BP-00133.

It is well known that ES cells are capable of differentiating into any type of cells. Therefore, the preantral follicle-derived ES cell of this invention may be a good source providing various types of cells. For example, the preantral follicle-derived ES cell may be induced to differentiate into hematopoietic cells, nerve cells, beta cells, muscle cells, liver cells, cartilage cells, epithelial cell, urinary tract cell and the like, by culturing it a medium under conditions for cell differentiation. Medium and methods which result in the differentiation of ES cells are known in the art as are suitable culturing conditions (Palacios, et al., PNAS. USA, 92:7530-7537(1995); Pedersen, J. Reprod. Fertil. Dev., 6:543-552(1994); and Bain et al., Dev. Biol, 168:342-357(1995)).

The preantral follicle-derived ES cell of this invention has numerous therapeutic applications through transplantation therapies. The preantral follicle-derived ES cell of this invention has application in the treatment of numerous diseases or disorders such as diabetes, Parkinson's disease, Alzheimer's disease, cancer, spinal cord injuries, multiple sclerosis, amyotrophic lateral sclerosis, muscular dystrophy, diabetes, liver diseases, i.e., hypercholesterolemia, heart diseases, cartilage replacement, bums, foot ulcers, gastrointestinal diseases, vascular diseases, kidney disease, urinary tract disease, and aging related diseases and conditions.

The present invention clearly demonstrates that ES cells can be derived from parthenogenetic activation of oocytes grown in in-vitro-cultured preantral (preferably, early secondary) follicles. In other words, immature (preantral) follicles allow to providing an alternative source of ES cells. The usefulness of preantral follicles as a source of ES cells can be elevated as long as suitable protocols of follicle culture, oocyte activation, embryo culture, and ES cell establishment are employed, as demonstrated in Examples. To our knowledge, this is the first invention on establishing homozygous ES cells without using somatic-cell nuclear transfer. This approach avoids the sacrifice both of ovulated oocytes having developmental competence and of viable embryos.

The present invention will now be described in further detail by examples. It would be obvious to those skilled in the art that these examples are intended to be more concretely illustrative and the scope of the present invention as set forth in the appended claims is not limited to or by the examples.

### Examples

### Materials and Methods

### I. Establishment of a Basic System for Manipulating Preantral Follicles

### Experimental Animals

Female F1 hybrid (C57BL6/DBA2) mice bred in the Laboratory of Embryology and Gamete Biotechnology, Seoul National University were maintained under controlled lighting (14L:10D), temperature (20 to 22° C.) and humidity (40 to 60%) and two-week-old sexually-immature (prepubertal) females were subsequently provided for this study. All procedures for animal management, breeding and surgery followed the standard operation protocols of Seoul National University. An Institutional Review Board, Department of Animal Science and Technology, Seoul National University approved our research proposal and relevant experimental procedures including animal care and use in October 2004. Appropriate management of experimental samples, and quality control of the laboratory facility and equipment were also conducted.

### Isolation of Preantral Follicles

The females were sacrificed by cervical dislocation and the ovaries were removed aseptically. For mechanical isolation of follicles, the ovaries were placed in 2 ml L-15 Leibovitz-glutamax medium (Sigma-Aldrich Corp, St. Louis, Mo.) supplemented with 10% (v/v) heat-inactivated fetal bovine serum (FBS) and 1% (v/v) lyophilized penicillin-streptomycin solution at 37° C. Two types of retrieval methods were employed for this study. Preantral follicles were retrieved mechanically by using a 30-gauge needle [5]. Otherwise, an enzymatic retrieval method was employed. In this method, the collected ovaries were placed in ribonucleoside and deoxyribonucleoside-containing α-MEM-glutamax medium supplemented with 0.1% (v/w) collagenase type I (198 units/mg; Sigma-Aldrich Corp.), 0.02% (v/w) DNase I (11.2 units/mg; Sigma-Aldrich Corp.) and 0.03% (v/v) fetal bovine serum (FBS) for 1 hr at 37° C. To facilitate proteolytic digestion, the ovaries were titrated every 30 min by gentle pipetting [6].

### Culture of Preantral Follicles

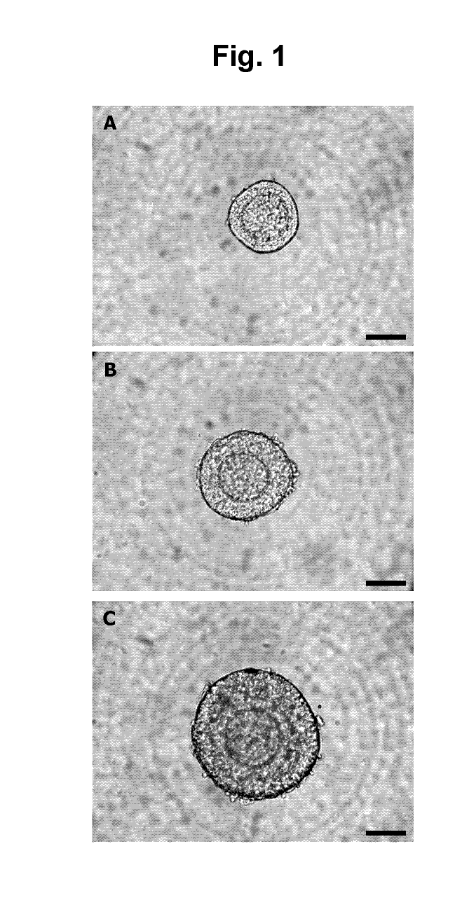

Preantral follicles isolated either mechanically or enzymatically were washed three times in 10 μl droplets of L-15 medium and subsequently classified into three categories by measuring diameter with an ocular micrometer of an inverted microscope (TE-2000; Nikon, Tokyo, Japan) at 40× magnification. The selection criteria are as follows: primary follicle of 75 to 99 μm, early secondary follicle of 100 to 125 μm and late secondary follicle of 126 to 180 μm in diameter. In addition to the size of the follicles, the typical morphology of the preantral follicles was employed for the classification (FIG. 1): primary follicles had a round follicular structure consisting of single compact layer of granulosa cells and a follicular oocyte. Early and late secondary follicles also had a round structure consisting of multiple layers of granulosa cells and a follicular oocyte. All categorized follicles were subsequently cultured at 37° C., 5% CO₂ in air atmosphere.

### In-Vitro Growth of Primary and Secondary Follicles

The primary follicles were placed singly in 10 μl culture droplets overlaid with washed-mineral oil (Sigma-Aldrich Corp.) in 60×15 mm Falcon plastic Petridishes (Becton Dickinson, Franklin Lakes, N.J.). The medium used for the culture of primary follicle is ribonucleoside and deoxyribonucleoside-free α-MEM-glutamax medium, to which 1% (v/v) heat-inactivated fetal bovine serum (FBS), 5 μg/ml insulin, 5 μg/ml transferrin, 5 ng/ml selenium, 100 mIU/ml recombinant human FSH (Organon, Oss, The Netherlands), 10 mIU/ml LH (cat. no. L-5259, Sigma-Aldrich Corp) and 1% (v/v) penicillin and streptomycin were added. On day 1 of culture, an additional 10 μl fresh medium was added to each droplet and half of a medium was changed everyday from day 3 of culture [5]. Cultured follicles were frequently detached from the bottom of culture dishes by mechanical pipetting. When the diameter of the follicles reached 100 μm (approximately on day 5 of culture), they were placed into 10 μl droplets of ribonucleoside and deoxyribonucleoside-containing α-MEM-glutamax medium supplemented with FBS, insulin, transferrin, selenium, FSH and antibiotics. On the next day, 10 μl of fresh medium was added to each droplet and, from the third day after the replacement, half of a medium was changed every other day [5].

The secondary follicles were also cultured individually and the culture protocol was similar to that for primary follicles except for only using ribonucleoside and deoxyribonucleoside-containing α-MEM-glutamax medium. Morphological change of preantral follicles was monitored everyday throughout the culture.

### Assessment of the Maturation of Follicular Oocytes

To induce maturation of follicular oocytes in preantral follicles, 2.5 IU/ml hCG (Pregnyl™; Organon) and 5 ng/ml epidermal growth factor (cat. no E-4127, Sigma-Aldrich Corp) were added to the culture medium 16 to 18 hr prior to the culture for oocyte maturation. Progress of meiotic maturation was monitored by staining oocytes with Lacmoid solution and the presence of germinal vesicle (GV) and GV breakdown (GVBD) in oocytes that did not have a first polar body was examined under a phase-contrast microscope. Oocyte maturation (developed to the metaphase II stage) was evaluated by the extrusion of the first polar body, and by mucification and expansion of cumulus cells enclosing oocyte. To monitor the extrusion of the first polar body, oocytes retrieved from cultured follicles were freed from cumulus cells by mechanical pipetting in M2 medium supplemented with 200 IU/ml hyaluronidase. The capacity of matured oocytes to form pronucleus to indirectly confirm cytoplasmic maturation was monitored after parthenogenetic activation using Ca²⁺-free KSOM medium supplemented with 10 mM SrCl₂ and 5 μg/ml cytochalasin B. The formation in activated oocytes was assessed by Hoechest staining under an inverted microscope equipped with a fluorescent apparatus. On the other hand, the size (diameter) and zona thickness of metaphase II (MII) stage oocytes derived from the cultured preantral follicles were also monitored under an inverted microscope equipped with an ocular micrometer.

### Statistical Analysis

A generalized linear model (PROC-GLM) in a Statistical Analysis System (SAS) program was employed and significant differences among treatments were determined where the P value was less then 0.05.

II. Homozygous Embryonic Stem Cells Derived from Preantral Follicles

### Experimental Animals

Two F1 hybrid strains were produced by mating female C57BL6 mice with male DBA2 or CBA/Ca mice. The established colonies were maintained in the Laboratory of Embryology and Gamete Biotechnology, Seoul National University, under controlled lighting (14L:10D), temperature (20-22° C.), and humidity (40-60%). Two-week-old prepubertal females were subsequently used in this study. All procedures for animal management, breeding, and surgery followed the standard protocols of Seoul National University. Appropriate management of experimental samples, and quality control of the laboratory facility and equipment were also conducted.

### Isolation of Early Secondary Follicles

The female mice were euthanized by cervical dislocation. The ovaries were removed aseptically and placed in 2 ml L-15 Leibovitz-glutamax medium (Gibco Invitrogen, Grand Island, N.Y.) supplemented with 10% (v/v) heat-inactivated fetal bovine serum (FBS; HyClone Laboratories, Logan, Utah) and 1% (v/v) lyophilized penicillin-streptomycin solution (Gibco Invitrogen) at 37° C. Subsequently, preantral follicles were retrieved mechanically using a 30-gauge needle [5]. Among the isolated preantral follicles, early secondary follicles, 100-125 μm in diameter with multiple layers of granulosa cells and an intrafollicular oocyte, were collected under the guidance of an ocular micrometer of an inverted microscope (TE-2000; Nikon, Tokyo, Japan) at 40× magnification. The follicles were washed three times in 10-μl droplets of L-15 medium and then cultured at 37° C. in an air atmosphere containing 5% CO₂.

### In Vitro Growth of Secondary Follicles

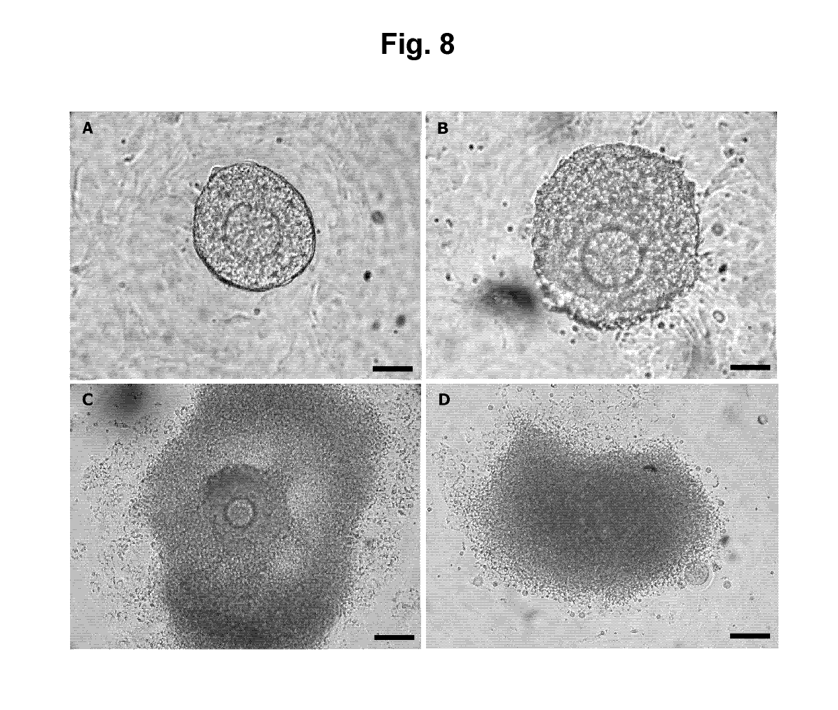

The retrieved follicles were placed singly in 10-μl culture droplets and then overlaid with washed mineral oil in 60×15 mm Falcon plastic Petri dishes (Becton Dickinson, Franklin Lakes, N.J.). Early secondary follicles were cultured in ribonucleoside- and deoxyribonucleoside-containing α-MEM-glutamax medium (Gibco Invitrogen) supplemented with 5% (v/v) FBS, 5 μg insulin/ml, 5 μg transferrin/ml, 5 ng selenium/ml, and 100 mIU recombinant human FSH (Organon, Oss, The Netherlands)/ml. All medium substrates were purchased from Sigma-Aldrich Corp. (St Louis, Mo.), unless otherwise stated. On day 1 of culture, an additional 10 μl of fresh medium was added to each droplet, and half of the medium was changed every other day from day 3 to the end of culture (Lenie et al., 2004). The morphological changes that occurred in the early secondary follicles during in vitro culture are depicted in FIG. 8.

### Collection of Mature Oocytes and Parthenogenetic Activation

Early secondary follicles 100-125 μm in diameter were cultured for 8-13 days, according to the experimental design; oocyte maturation was triggered by exposure to 2.5 IU human chorionic gonadotrophin (hCG) (Pregnyl; Organon, Oss, The Netherlands)/ml and 5 ng epidermal growth factor/ml at 16 hr before the end of culture. Maturation of the oocytes to the metaphase II stage was determined by extrusion of the first polar body and by detecting mucification and expansion of cumulus cells. Oocytes were freed from cumulus cells by mechanical pipetting in M2 medium, consisting of 94.66 mM NaCl, 4.78 mM KCl, 1.71 mM CaCl₂.2H₂O, 1.19 mM KH₂PO₄, 1.19 mM MgSO₄.7H₂O, 4.15 mM NaHCO₃, 20.85 mM HEPES, 23.28 mM sodium lactate, 0.33 mM sodium pyruvate, 5.56 mM glucose, 1% (v/v) penicillin/streptomycin, and 4 mg bovine serum albumin (BSA)/ml, supplemented with 200 IU hyaluronidase/ml. Mature oocytes were activated parthenogenetically by culturing for 4 h in Ca²⁺-free KSOM medium supplemented with 10 mM SrCl₂ and 5 μg/ml cytochalasin B.

### Culture of Activated Oocytes

Modified Chatot, Ziomek, and Bavister (CZB) medium was used for the culture of parthenogenetically activated oocytes. CZB consists of 81.6 mM NaCl, 4.8 mM KCl, 1.2 mM KH₂PO₄, 1.2 mM MgSO₄.7H₂O, 1.7 mM CaCl₂.2H₂O, 25.1 mM NaHCO₃, 31.3 mM sodium lactate, 0.3 mM sodium pyruvate, 1 mM glutamine, 0.1 mM EDTA, and 5 mg BSA/ml. Subsequently, 0.001 mg Hb (methemoglobin type)/ml and 5.5 μM β-mercaptoethanol (Gibco Invitrogen) were added to the CZB medium. Activated oocytes were cultured for about 5 days in a 5-μl droplet of medium overlaid with washed mineral oil at 37° C. in an air atmosphere containing 5% CO₂ (Lee et al., 2004). Development of activated oocytes to the blastocyst stages was monitored under either a stereomicroscope (SMZ-3; Nikon, Tokyo, Japan) or an inverted microscope (Eclipse TE-3000; Nikon) at about 140 hr after hCG injection.

### Establishment of ES Cells

The zona pellucida of collected blastocysts were removed using acid Tyrode solution, and the zone-free blastocysts were subsequently cultured on a feeder layer of mouse embryonic fibroblasts (MEFs) treated with 10 μg mitomycin C (Chemicon, Temecula, Calif.)/ml for 3 hr in gelatin-coated four-well multi-dishes. Knock-out Dulbecco's minimal essential medium (KDMEM; Gibco Invitrogen) supplemented with 0.1 mM β-mercaptoethanol (Gibco Invitrogen), 1% (v/v) nonessential amino acids (Gibco Invitrogen), 2 mM L-glutamine, a 1% (v/v) lyophilized mixture of penicillin and streptomycin, and 2,000 units mouse LIF (Chemicon)/ml, and a 3:1 mixture of FBS and knock-out serum replacement were used for initial culture of the blastocysts. On day 4 of culture, inner cell mass (ICM) cell-derived cell colonies were mechanically removed with a capillary pipette and replated on the MEF feeder for further expansion. Expanded colonies were dissociated with 0.04% (v/w) trypsin-EDTA (Gibco Invitrogen) and subcultured on a 35-mm tissue culture dish in the presence or absence of MEF feeder cells under a humidified atmosphere of 5% CO₂ in air at 37° C. Subpassage was conducted at 4-day intervals, when the cultured ES cells had reached 70-80% confluency. The medium was changed daily during subculture.

### Chromosome Analysis

The chromosomes of established ES cells were analyzed at 20 subpassages. ES cells were incubated in culture medium supplemented with 0.1 μg colcemid/ml for 3 h at 37° C. in an atmosphere of 5% CO₂ in air. The treated cells were trypsinized, resuspended for 15 min in 0.075 M KCl at 37° C., placed in hypotonic solution, and subsequently fixed in a 3:1 (v/v) mixture of methanol and acetic acid. Chromosomes were spread onto heat-treated slides and then stained with Giemsa solution.

### Marker Assay

ES cell colonies collected from the twentieth subpassage were washed with PBS (Gibco Invitrogen) containing Ca²⁺ and Mg²⁺, fixed in 4% (v/v) formaldehyde at room temperature for 10 min, washed twice with the PBS, and then stained with alkaline phosphatase (AP). Reactive colonies were visualized with fast red TR/naphthol AS-MX phosphate. Staining with anti-stage-specific embryonic antigen (SSEA)-1 (MC-480, 1:1000 dilution), anti-SSEA-3 (MC-631, 1:1000 dilution), anti-SSEA-4 (MC-813-70, 1:1000 dilution), anti-integrin α6 (P2C62C4, 1:1000 dilution), and anti-integrin β1 (MH25, 1:1000 dilution) antibodies was carried out using monoclonal antibodies supplied by the Developmental Studies Hybridoma Bank (Iowa City, Iowa). Localization of the antibodies was detected using the DakoCytomation kit (DakoCytomation, Carpinteria, Calif.).

Embryonic Body Formation and Detection of Cells Originating from the Three Germ Layers

Established ES cells were transferred into 100-mm plastic Petri dishes after treatment with 0.04% (v/v) trypsin-EDTA solution (Gibco Invitrogen). The cell suspension was cultured in LIF- and β-mercaptoethanol-free culture medium until embryoid bodies formed. Each embryoid body was then seeded into 96-well culture plates, cultured for 7 days, and then stained with markers specific for the three germ layers: neural cadherin adhesion molecule (NCAM, 1:1,000 dilution; BIODESIGN International, Saco, Me.) and S-100 (1:1000 dilution; BIODESIGN International) for the ectodermal layer; muscle actin (1:1000 dilution; BIODESIGN International) and desmin (1:1000 dilution; Santa Cruz Biotechnology, Delaware, Calif.) for the mesodermal layer; and α-fetoprotein (1:1000 dilution; BIODESIGN International) and Troma-1 (1:1000 dilution; Hybridoma Bank) for endodermal cells. Antibody localization was detected as noted above.

### Induction and Detection of Neuronal Differentiation

For in vitro-differentiation into neuronal lineage cells, undifferentiated ES cells were dissociated and plated onto 0.1% gelatin-coated plastic culture dish at a density of 0.5-1.5×10⁴/cm², which contained in modified N2B27 medium consisting of DMEM/F12 supplemented with N2 (Gibco Invitrogen) and B27 (Gibco Invitrogen). Culture with morphological evaluation was continued for 1 week and the medium was renewed at 2-day intervals. For cell maintenance, the differentiated cells were replated onto fibronectin coated tissue culture dish.

Immunohistochemical analysis was conducted to detect cell differentiation. Differentiated cells were fixed with 4% paraformaldehyde for 5 minutes. After blocking with PBS supplemented with 5% FBS, the fixed cells were reacted with primary antibodies: Nestin (goat IgG, SC-21247, Santa Cruz Biotechnology), β-tubulin type III (mouse IgG, CBL412, Chemicon, Temecula, Calif.), O4 (mouse IgM, MAB345, Chemicon) and GFAP (mouse IgG, MAB360, Chemicon). The antigen-antibody complexes were visualized with fluorescent secondary antibodies: Alexa Fluor 488-conjugated anti-goat IgG (A-11055, Molecular Probes, Eugene, Oreg.), Alexa Fluor 568-conjugated anti-mouse IgG (A11061, Molecular Probes) or Alexa Fluor 488-conjugated anti-mouse IgM (A-21042, Molecular Probes). The stained cells were observed under a laser scanning confocal microscope with a krypton-argon mixed gas laser excitation at 488 nm or 568 nm, and a fluorescein filter (Bio-Rad, Hemel Hempstead, UK).

### Teratoma Formation

Established ES cells maintained for up to 20 passages on MEF feeder layers were harvested in the absence of feeder cells, and 1×10⁷ cells were injected subcutaneously into adult NOD-SCID mice. Teratomas retrieved 8 weeks post-injection were fixed in 4% (v/v) paraformaldehyde. The tissues were embedded in a paraffin block, stained with hematoxylin and eosin, and examined under a phase-contrast microscope (BX51TF; Olympus, Kogaku, Japan).

### Deposit of Homozygous Preantral Follicle-Derived ES Cell

Of the follicle-derived ES cells showing all of the ES cell characteristics described above, one cell was named “FpB6D2-snu-1” and deposited on Apr. 10, 2006 in the International Depository Authority, the Korean Cell Line Research Foundation and was given accession No. KCLRF-BP-00133.

### Results

### I. Establishment of a Basic System for Manipulating Preantral Follicles

### Comparison of Retrieval Efficiency

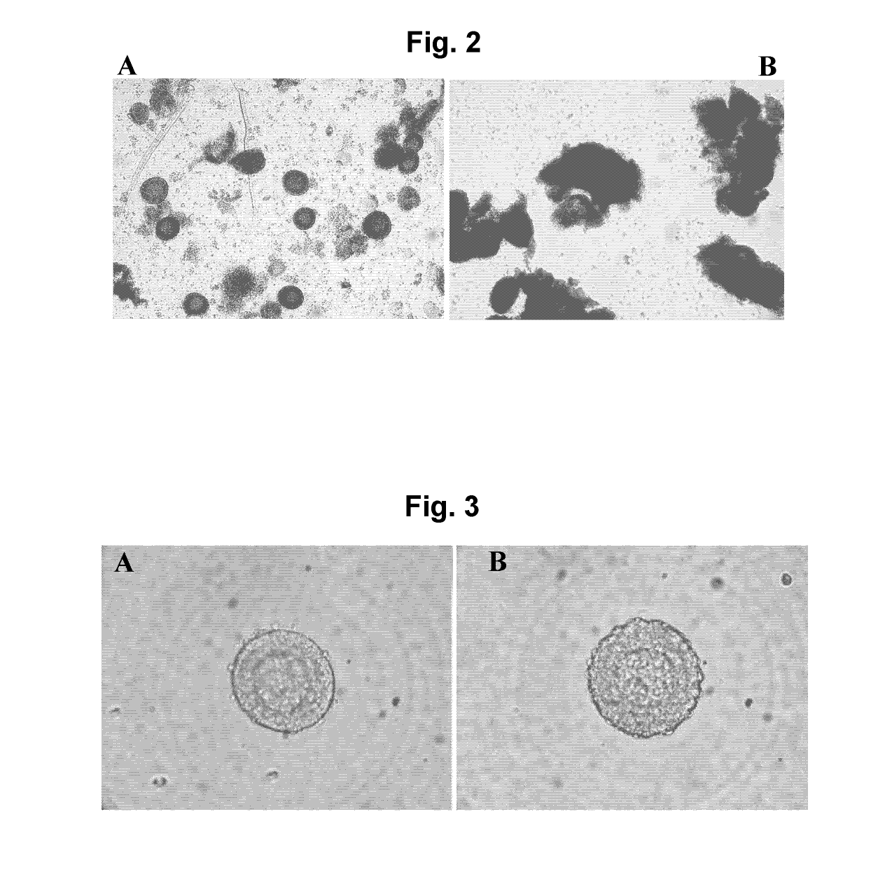

Total 2,432 preantral follicles were retrieved from the ovaries by two different methods (Table I). When cell population was compared at retrieval, the number of early secondary follicles was larger (P<0.0001) than that of primary and late secondary follicles (1,249 cells vs. 485 to 698 cells), regardless of the retrieval methods. As shown in FIG. 2, the preantral follicles collected from the ovaries were present singly or in groups. In the case of follicles being collected in groups, it is very difficult to separate single follicles from the complexes and accordingly the single culture of the follicles collected in groups was not possible.

Overall, the total number of preantral follicles retrieved per mouse was larger (P<0.0001) when using the mechanical method than when using the enzymatic method (339±48 cells vs. 202±28 cells). Due to the enzyme treatment, the degree to which preantral follicles aggregated to each other was very low. The number of primary, early secondary and late secondary follicles retrieved in groups by the mechanical method was 84±14, 97±12 and 56±17 cells, respectively. The enzymatic method yielded more (P<0.0001) preantral follicles collected as a single complex than the mechanical method (202±28 cells vs. 102±26 cells): an increased number of primary (52±12 cells vs. 35±9 cells), early secondary (110±18 cells vs. 46±13 cells) and late secondary (39±12 cells vs. 21±7 cells) follicles in the enzymatic retrieval was detected.

As shown in FIG. 3, the preantral follicles retrieved by the mechanical method had a spherical shape and their basement membrane remained intact. Few theca cells still attached with the basement membrane. The preantral follicles retrieved by the enzymatic method lost the basement membrane partly or wholly and the theca cells no longer attached in the follicles. The cytoplasm, especially in the marginal region, of the preantral follicles retrieved by the enzymatic method became coarse compared with that of the follicles collected by the mechanical method.

TABLE IA
Retrieval of preantral follicles of different stages (primary, early
secondary and late secondary) by either a mechanical or an enzymatic
(use of collagenase and DNAase) method

| | Total | | | | |
|---|---|---|---|---|---|
| | mean ± SD | Mean ± SD number of preantral | | | |
| | number of | follicles retrieved singly | | | |
| Isolation | follicles | | Early | Late | Subtotal |
| methods | retrieved | Primary | secondary | secondary | number |
| Mechanical | 339 ± 48^{a} | 35 ± 9 | 46 ± 13 | 21 ± 7 | 102 ± 26^{a} |
| Enzymatical | 202 ± 28^{b} | 52 ± 12 | 110 ± 18 | 39 ± 12 | 202 ± 28^{b} |

Total 16 female F1 mice were sacrificed and each treatment replicated 8 times.
Model effects in the total number of preantral follicles retrieved, subtotal number of the follicles retrieved singly and in group were less than 0.0001 (P values).

TABLE IB
Retrieval of preantral follicles of different stages (primary, early
secondary and late secondary) by either a mechanical or an enzymatic
(use of collagenase and DNAase) method

| | Total | | | | |
|---|---|---|---|---|---|
| | mean ± SD | Mean ± SD number of preantral | | | |
| | number of | follicles retrieved in groups | | | |
| Isolation | follicles | | Early | Late | Subtotal |
| methods | retrieved | Primary | secondary | Secondary | number |
| Mechanical | 339 ± 48^{a} | 84 ± 14 | 97 ± 12 | 56 ± 17 | 237 ± 38^{a} |
| Enzymatical | 202 ± 28^{b} | 0 | 0 | 0 | 0^{b} |

Total 16 female F1 mice were sacrificed and each treatment replicated 8 times.
Model effects in the total number of preantral follicles retrieved, subtotal number of the follicles retrieved singly and in group were less than 0.0001 (P values).

### Morphological Change During Culture of Preantral Follicle

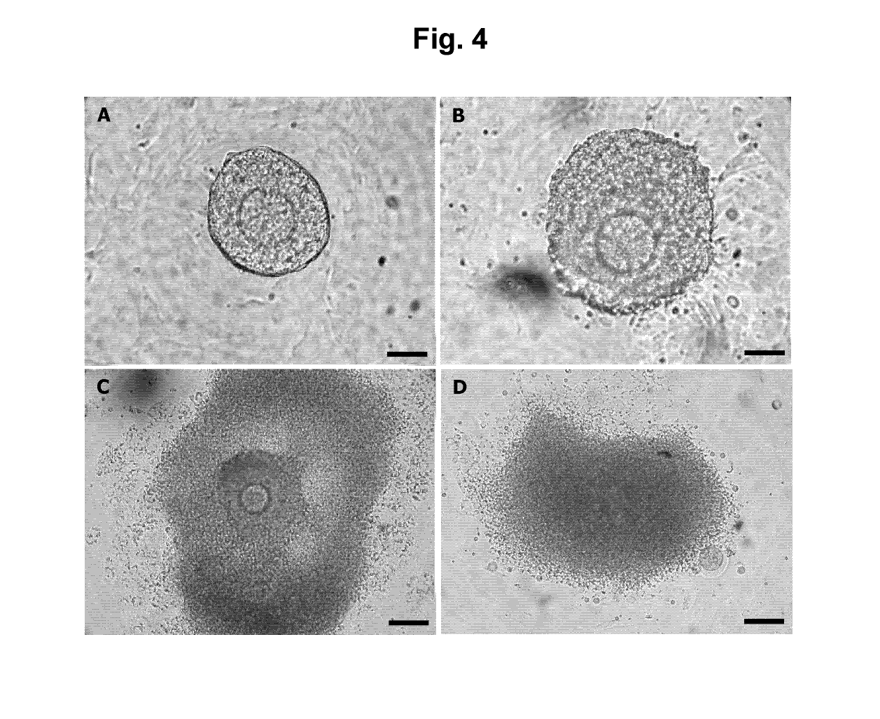

In general, in vitro-growth of preantral follicles was classified into four stages, namely the follicular, diffuse, pseudoantral and degenerative stages (FIG. 4). The preantral follicles in the follicular stage remained intact morphology, which had spherical and a distinct basement membrane. At the diffuse stage, the granulosa cells enclosing the follicular oocyte vigorously proliferated, which induced the expansion and multiplication of granulosa cell layers. The increase of follicle size was eminently detected compared with the follicular stage. The preantral follicles at the pseudoantral stage were characterized as forming antrum-like, granulosa cell-free area. Maximal expansion of granulosa cells allow creation of an empty space between the granulosa cell matrix, and the basement membrane of the follicle was no longer visible. Intrafollicular oocyte and its adjacent granulosa (cumulus) cells spontaneously dispatched (released) from the cell complex. At the degenerative stage, black spots were visible in granulosa cell matrix. The viability of granulosa cells gradually decreased, which finally led the breakdown of the granulosa cell complex.

### In Vitro-Growth of Preantral Follicles

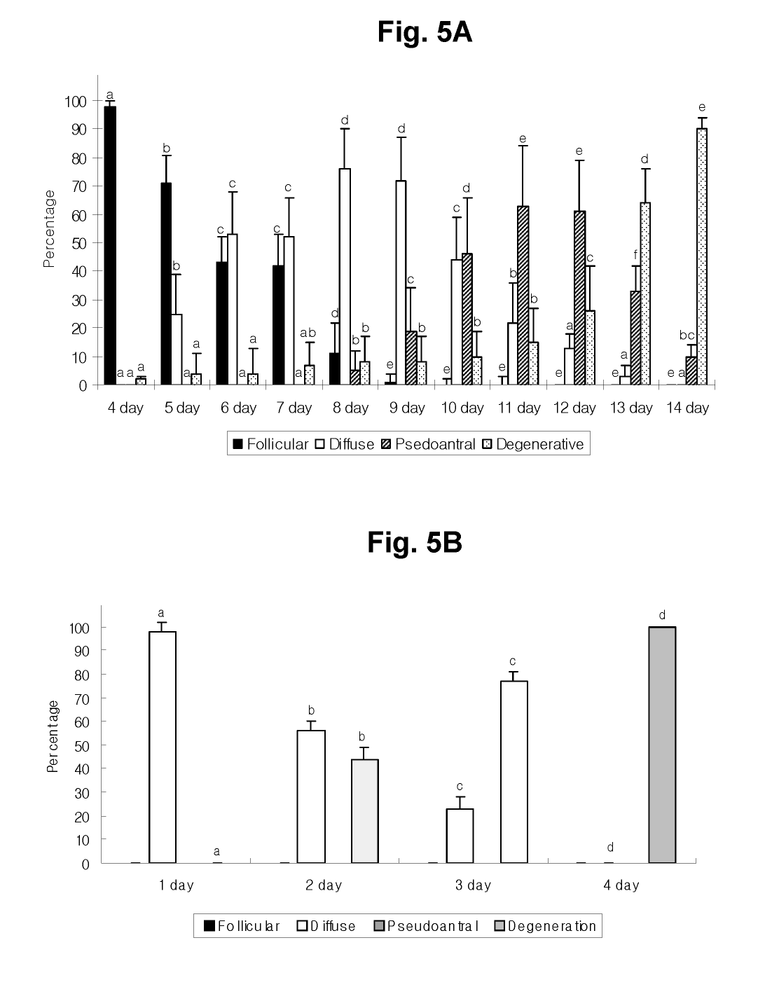

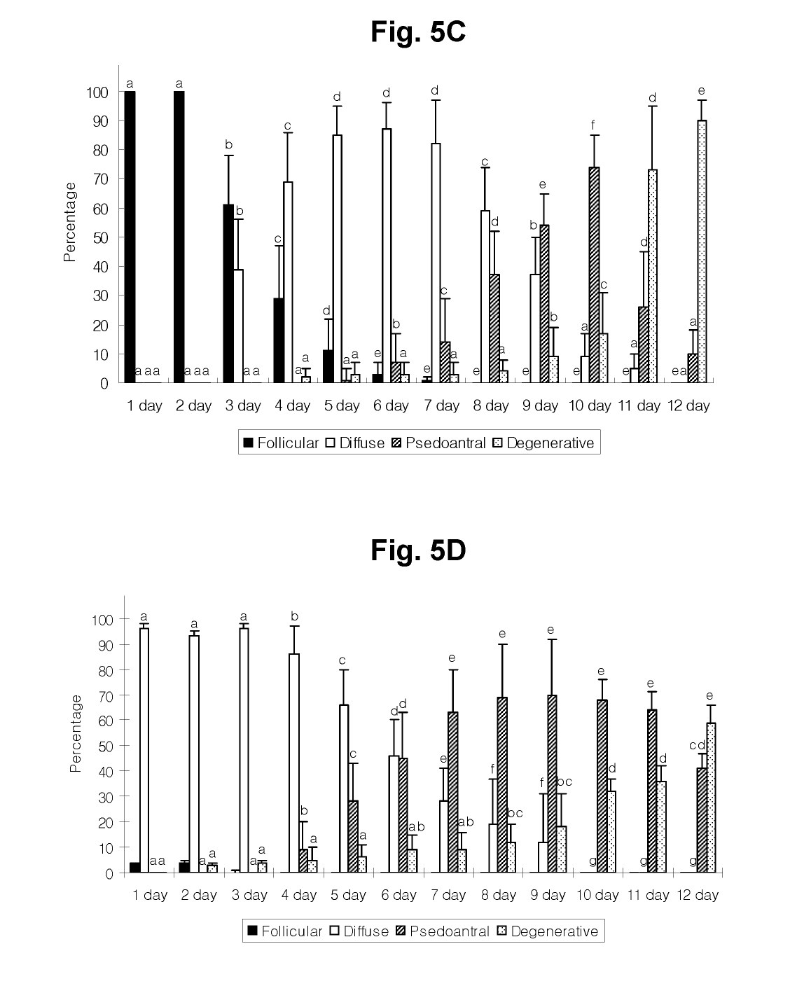

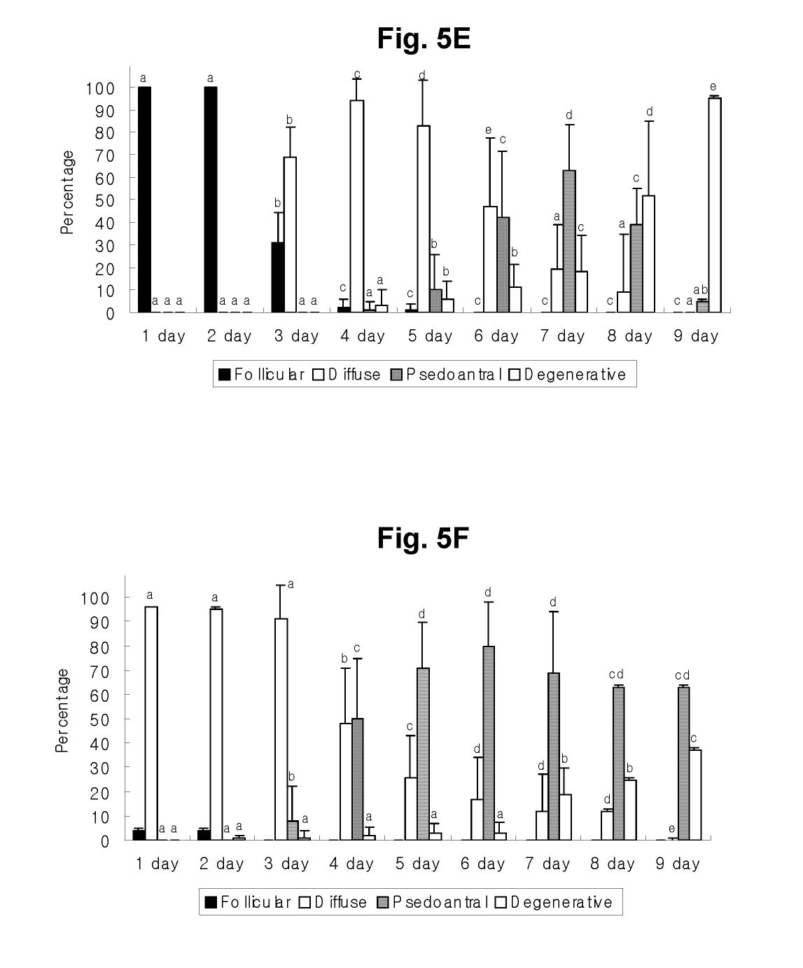

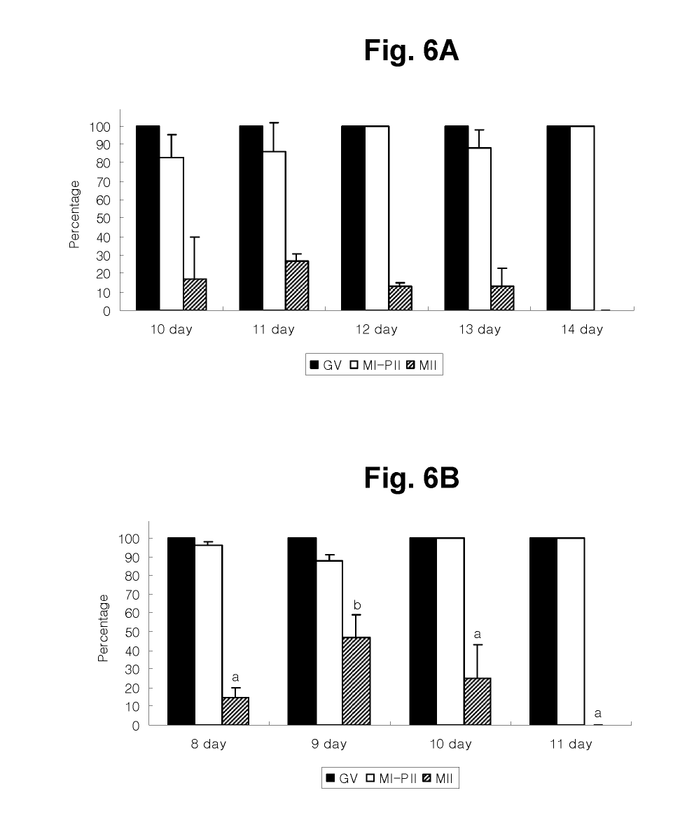

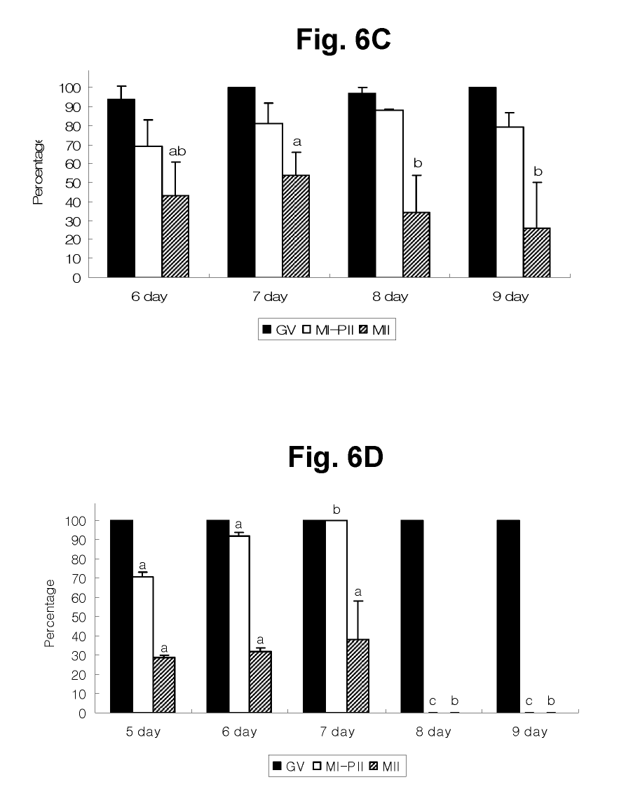

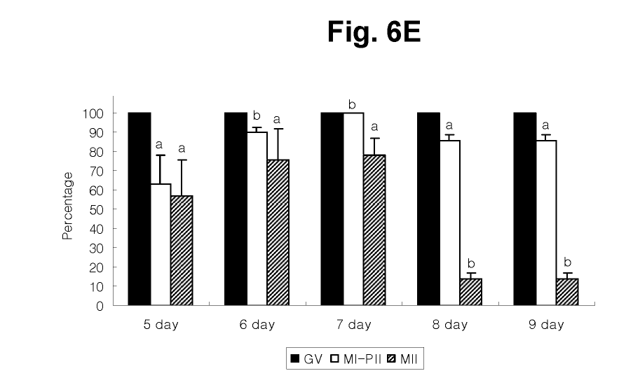

Regardless of the types of preantal follicles, all follicles cultured in vitro went through a step-by-step growth from the follicular to degenerative stages. As shown in FIG. 5, there were significant differences in in vitro-growth of preantal follicles, and both the developmental stage of preantral follicle and the retrieval method affected the growth. In the case of primary follicles, the follicles collected by a mechanical method entered the diffuse stage on day 6 of culture. The primary follicles entered into the pseudoantral stage from day 8 (5%) of culture and peaked incidence was on day 11 (63%). The degenerative stage was detected throughout the observation period (day 8 to day 14 of culture). Major proportion (97%) of primary follicles retrieved by the enzymatic method entered into the diffuse stage on day 1 of culture (97%). However, no follicles developed into the pseudoantral stage and all of the follicles become degenerated by day 4 of culture. In the case of early secondary follicles, the pseudoantral stage was firstly detected on day 5 and on day 4 of culture in mechanical and enzymatic retrieval, respectively. The incidence of the follicles developed into the diffuse and pseudoantral stage was peaked on day 6 (87%) and day 10 (74%) of culture in the case if mechanical retrieval, respectively, while on day 4 (86%) and 9 (70%) of culture in the the case of enzymatic retrieval. In the case of late secondary follicles, the incidence of the diffuse stage was peaked on day 4 (94%) of culture in the mechanical retrieval and day 3 (91%) of culture in the enzymatic retrieval. The follicles developed into the pseudoantral stage first appeared on day 4 (1%) and day 3 (8%) of culture in the group of mechanical and enzymatic retrieval, respectively. The incidence was peaked on day 7 (63%) and day 6 (80%) of culture, respectively.

### Maturation and Fertilizability of Follicular Oocytes

Since no primary follicles retrieved by an enzymatic method developed into the pseudoantral stage (FIG. 5), oocytes derived from total 5 categories of follicles (primary follicles retrieved by a mechanical method, early and late secondary follicles retrieved mechanically or enzymatically) were provided for this experiment (FIG. 5). In the case of primary oocytes, MII stage oocytes first appeared on day 10 (17%) and peaked on day 11 (27%) of culture. On the other hand, oocytes retrieved from early secondary follicles reached the MII stage from day 8 (15%) and day 6 (43%) of culture in the mechanical and the enzymatic method, respectively. The optimal time to retrieve MII stage oocytes was on day 9 (47%) in the mechanical and day 7 (54%) of culture in the enzymatic method. In the case of late secondary follicles, oocytes reached the MII stage from day 5 (29% in the mechanical and 57% in the enzymatic) of culture in each method and the peak time of oocyte maturation was day 7 (38% in the mechanical and 78% in the enzymatic) of culture.

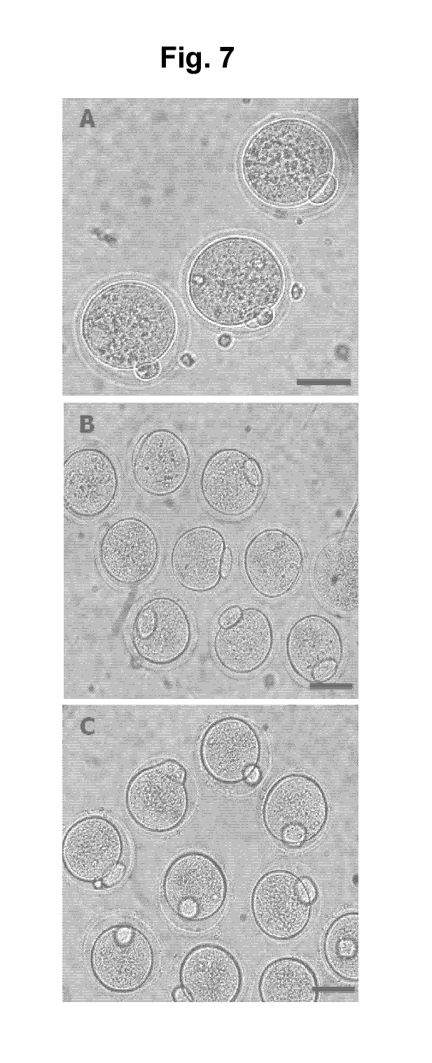

The zona thickness and the diameter of MII stage oocytes retrieved from in vitro-cultured preantral follicles were compared with those of oocytes ovulated in vivo. As shown in Table II and FIG. 7, oocyte diameter was generally decreased in all groups of oocytes derived from in vitro-cultured preantral follicles compared with in vivo-derived oocytes (63.31 to 65.53 μm vs. 75 μm). A significantly lower thickness was specifically detected in oocytes derived from the enzymatically retrieved follicles (5.41 to 5.74 μm vs. 7.76 μm). Oocytes derived from the primary follicles had smaller diameters than oocytes derived from the early and the late secondary follicles.

The rate of pronuclear formation after parthenogenetic activation was within the range of 86 to 94% (Table III) and 91% of in vivo-derived oocytes formed pronucleus after the activation. No significant difference among the treatments was detected.

TABLE II
Effects of follicle retrieval methods on the thickness of zona pellucida
and the diameter of metaphase II (MII) stage oocytes grown in in vitro-
cultured primary, early secondary or late secondary follicles

| | Retrieval | No. of | Mean | Mean |
|---|---|---|---|---|
| | method for | MII stage | thickness | diameter |
| Origin of | in vitro | oocytes | (μm) of zona | (μm) of |
| oocytes | culture | evaluated | pellucida | oocytes |
| Primary | Mechanical | 52 | 7.88 ± 1.06^{a} | 63.31 ± 3.35^{a} |
| follicle | | | | |
| Early | Mechanical | 52 | 8.08 ± 0.91^{a} | 64.57 ± 2.60^{b} |
| secondary | | | | |
| follicle | | | | |
| Early | Enzymatic | 52 | 5.74 ± 0.74^{b} | 65.20 ± 1.92^{b} |
| secondary | | | | |
| follicle | | | | |
| Late | Mechanical | 52 | 7.65 ± 0.78^{a} | 65.06 ± 3.21^{b} |
| secondary | | | | |
| follicle | | | | |
| Late | Enzymatic | 52 | 5.41 ± 0.89^{b} | 65.53 ± 2.40^{b} |
| seconday | | | | |
| follicle | | | | |
| Graffian | — | 20 | 7.76 ± 0.16^{a} | 75.0 ± 0.04^{c} |
| follicle | | | | |
| (in vivo) | | | | |

Model effects in the thickness of zona pellucida and the diameter of oocytes were less than 0.0001 (P values).
^{abc}Different superscripts within a column are significantly different, P < 0.05.

The rate of pronuclear formation after parthenogenetic activation was within the range of 86 to 94% (Table III) and 91% of in vivo-derived oocytes formed pronucleus after the activation. No significant difference among the treatments was detected.

TABLE III
Formation of pronucleus after the parthenogenetic activation
of mature oocytes derived from primary, early secondary or late
secondary follicles^{a} cultured in vitro

| | Methods of | | | |
|---|---|---|---|---|
| | preantral | Stages of the | | |
| Oocytes | follicle | follicles | No. (%) of MII stage oocytes | |
| matured | Retrieval | retrieved | Activated^{c} | Formed pronuclei |
| In-vivo^{b} | N/A | N/A | 45 | 41 (91) |
| In-vitro | Mechanical | Primary | 14 | 12 (86) |
| | | Early secondary | 23 | 21 (91) |
| | | Late secondary | 16 | 15 (94) |
| | Enzymatic | Early secondary | 57 | 53 (93) |
| | | Late secondary | 49 | 45 (92) |

^{a}Preantral follicles cultured were retrieved from the ovaries by two different methods.
^{b}Oocytes were collected from the oviduct flushing after natural ovulation.
^{c}Parthenogenetic activation was conducted by the treatment with SrCl₂ and cytochalasin B.
Model effects in the number of MII oocytes to form pronuclei was 0.972 (P values).

II. Homozygous Embryonic Stem Cells Derived from Preantral Follicles
Manipulation of Preantral Follicles Derived from F1 (C57BL6×DBA2) Hybrid Mice

Preliminary experiments showed that approximately 60% of the preantral follicles retrieved mechanically were early secondary follicles, while the remaining 40% were either primary (<100 μm in diameter) or late secondary (>125 μm in diameter) follicles. When mature oocytes cultured for 8-10 days were treated with strontium chloride and cytochalasin B, more than 90% were parthenogenetically activated to form two pronuclei, regardless of the culture duration. However, neither the 8-day nor the 10-day culture yielded cleaved oocytes after being parthenogenetically activated. As shown in Table IV, the 9-day culture yielded optimal cleavage (29/107=27%), but the addition of LH at any dose to the culture medium did not further improve cleavage rates (16-33%). Of the 13 replicates, 25 blastocysts were derived from 116 oocytes, and one primary ES cell line was established by culturing in LIF-containing medium.

Manipulation of Preantral Follicles Derived from F1 (C57BL6×CBA/Ca) Hybrid Mice

Based on the results from C57BL6×DBA2 mice, LH was not added to the follicle culture medium. Intrafollicular oocytes in the preantral follicles were cultured for 8-13 days, and the rate of cleavage after parthenogenetic activation was 43% (3/7), 67% (61/92), 33% (4/12), 50% (6/12), 0% (0/7), and 0% (0/11) for 8-, 9-, 10-, 11-, 12-, and 13-day cultures, respectively (Table IV). Of 74 cleaved oocytes derived from five replicates, 59 (80%) developed into blastocysts. Nine primary ES cell lines were established, all of which were derived from oocytes cultured for 9 days.

TABLE IV
Accumulative data on the establishment of embryonic stem (ES) cells
derived from different mouse hybrid strains (C57BL/DBA2 and C57BL/CBAca)

| | | | | | No. (%)^{c} of oocytes | | | |
|---|---|---|---|---|---|---|---|---|
| | | Time | | No. of | | Developed | No. of | No. of ES |
| | | of | Medium^{b} | oocytes | | to | ICM cells | cells |
| Strains | Sets | retrieval^{a} | supplements | activated | Cleaved | blastocysts | colonized | established |
| B6/D2 | 1 | 8 | None | 1 | 0 (0) | 0 (0) | 0 | 0 |
| | 1 | 8 | None | 3 | 0 (0) | 0 (0) | 0 | 0 |
| | 1 | 10 | None | 3 | 0 (0) | 0 (0) | 0 | 0 |
| | 2 | 9 | None | 19 | 10 (53) | 4 (21) | 0 | 0 |
| | 2 | 9 | 2.5 IU LH | 17 | 7 (41) | 2 (12) | 0 | 0 |
| | 2 | 9 | 5 IU LH | 16 | 13 (81) | 2 (13) | 0 | 0 |
| | 3 | 9 | 2.5 IU LH | 15 | 6 (40) | 2 (13) | 1 | 1 |
| | 3 | 9 | 5 IU LH | 15 | 5 (33) | 1 (7) | 0 | 0 |
| | 3 | 9 | 10 IU LH | 16 | 1 (6) | 0 (0) | 0 | 0 |
| | 4 | 9 | None | 5 | 5 (100) | 3 (60) | 0 | 0 |
| | 4 | 9 | 5 IU LH | 12 | 8 (67) | 3 (25) | 0 | 0 |
| | 4 | 9 | 10 IU LH | 12 | 7 (58) | 0 (0) | 0 | 0 |
| | 5 | 9 | None | 14 | 0 (0) | 0 (0) | 0 | 0 |
| | 5 | 9 | 5 IU LH | 7 | 0 (0) | 0 (0) | 0 | 0 |
| | 5 | 9 | 10 IU LH | 15 | 0 (0) | 0 (0) | 0 | 0 |
| | 6 | 9 | None | 18 | 0 (0) | 0 (0) | 0 | 0 |
| | 6 | 9 | 5 IU LH | 13 | 0 (0) | 0 (0) | 0 | 0 |
| | 6 | 9 | 10 IU LH | 17 | 1 (6) | 0 (0) | 0 | 0 |
| | 7 | 9 | None | 12 | 0 (0) | 0 (0) | 0 | 0 |
| | 7 | 9 | 5 IU LH | 13 | 1 (8) | 1 (8) | 0 | 0 |
| | 7 | 9 | 10 IU LH | 14 | 0 (0) | 0 (0) | 0 | 0 |
| | 8 | 9 | None | 9 | 2 (22) | 0 (0) | 0 | 0 |
| | 8 | 9 | 2.5 IU LH | 14 | 3 (21) | 0 (0) | 0 | 0 |
| | 8 | 9 | 5 IU LH | 14 | 3 (21) | 0 (0) | 0 | 0 |
| | 9 | 9 | 2.5 IU LH | 8 | 4 (50) | 2 (25) | 0 | 0 |
| | 9 | 9 | 5 IU LH | 10 | 2 (20) | 0 (0) | 0 | 0 |
| | 9 | 9 | 10 IU LH | 8 | 4 (50) | 0 (0) | 0 | 0 |
| | 10 | 9 | None | 10 | 5 (50) | 0 (0) | 0 | 0 |
| | 10 | 9 | 2.5 IU LH | 15 | 3 (20) | 3 (20) | 0 | 0 |
| | 10 | 9 | 10 IU LH | 13 | 3 (23) | 0 (0) | 0 | 0 |
| | 11 | 9 | None | 12 | 4 (33) | 0 (0) | 0 | 0 |
| | 11 | 9 | 2.5 IU LH | 10 | 5 (50) | 1 (10) | 0 | 0 |
| | 11 | 9 | 10 IU LH | 9 | 1 (11) | 0 (0) | 0 | 0 |
| | 12 | 9 | None | 8 | 3 (38) | 0 (0) | 0 | 0 |
| | 12 | 9 | 2.5 IU LH | 11 | 4 (36) | 0 (0) | 0 | 0 |
| | 12 | 9 | 10 IU LH | 12 | 1 (8) | 1 (8) | 0 | 0 |
| | 13 | 9 | 2.5 IU LH | 17 | 3 (18) | 0 (0) | 0 | 0 |
| | 13 | 9 | 5 IU LH | 8 | 2 (25) | 0 (0) | 0 | 0 |
| | Total | Optimal retrieval time = 9 days after culture/1 ES cell | | | | | | |
| | | line from 25 blastocysts (9 replicates) | | | | | | |
| B6C/Ca | 1 | 9 | None | 13 | 9 (69) | 7 (54) | 3 | 1^{d} |
| | 2 | 9 | None | 68 | 42 (62) | 37 (54) | 17 | 7^{d} |
| | 3 | 8 | None | 7 | 3 (43) | 1 (14) | 1 | 0 |
| | 3 | 9 | None | 11 | 10 (91) | 8 (73) | 4 | 1^{d} |
| | 4 | 10 | None | 12 | 4 (33) | 2 (17) | 2 | 0 |
| | 4 | 11 | None | 12 | 6 (50) | 4 (33) | 1 | 0 |
| | 5 | 12 | None | 7 | 0 (0) | 0 (0) | 0 | 0 |
| | 5 | 13 | None | 11 | 0 (0) | 0 (0) | 0 | 0 |

| | |
|---|---|
| Total | Optimal retrieve time = 9 days after culture/more than 9 ES cell |
| | line from 59 blastocysts (5 replicates) |

^{a}Duration of culture for retrieving pseudoantral follicles.
^{b}Ribonucleoside- and deoxyribonucleoside-containing α-MEM-glutamax medium supplemented with FBS, insulin, transferrin, selenium, and recombinant human FSH was used as a based medium for the culture of early secondary follicles.
^{c}Percentage of the number of oocytes activated artificially with SrCl₂ and cytochalasin B.
^{d}Rest of colony-forming ICM cell batches were stored at −196° C.

### Characterization of ES Cells

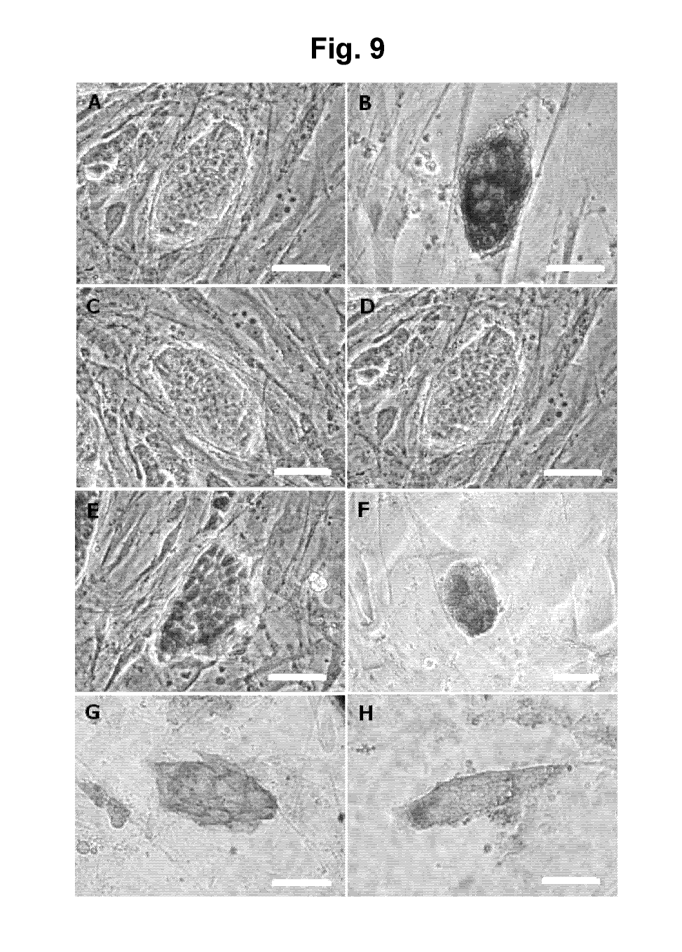

Ten primary ES cell cultures (1 from C57BL6×DBA2 mice and 9 from C57BL6×CBA/Ca mice) were established and the established cells were successfully subcultured more than 50 times except one line derived from C57BL6×CBA/Ca. Colony-forming cells at the 20^{th} subpassage stained positively for AP, anti-SSEA-1, anti-integrin α6, anti-integrin β1, and Oct-4 antibody, whereas no reactivity to anti-SSEA-3 or anti-SSEA-4 antibodies was detected (FIG. 9).

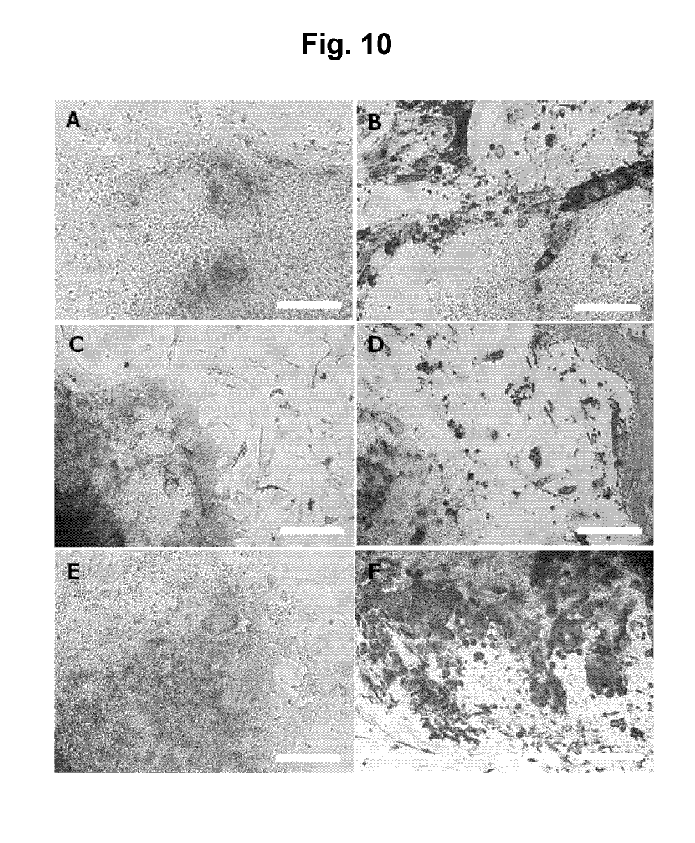

The established cells subsequently formed embryoid bodies in the absence of LIF. Immunocytochemical analysis showed that the embryoid-body-forming cells were positive for markers specific for one of the three germ layers. Neural cadherin adhesion molecule, S-100, Troma-1, muscle actin, desmin, and α-fetoprotein were used as markers (FIG. 10).

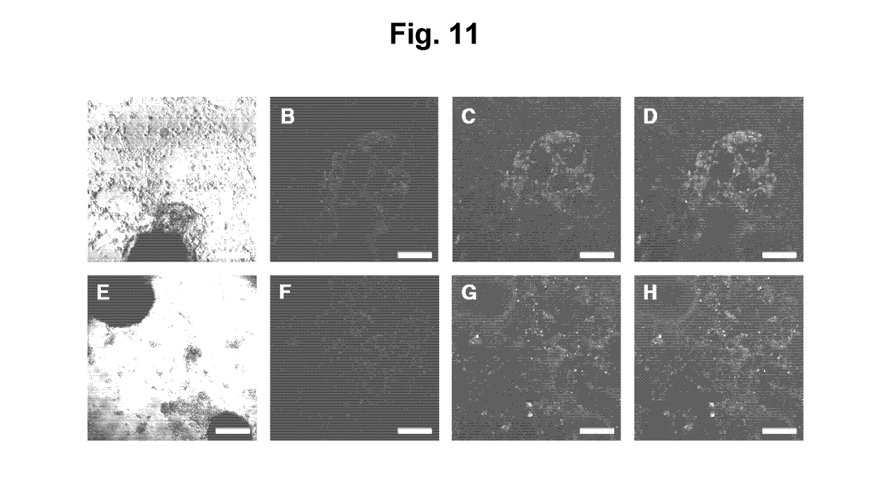

As shown in FIG. 11, the established cells further differentiated into neurons (Tuj1- and nestin-positive cells), oligodendrocytes (O4-positive cells) and astrocytes (GFAP-positive cells) after cultured in the designated medium.

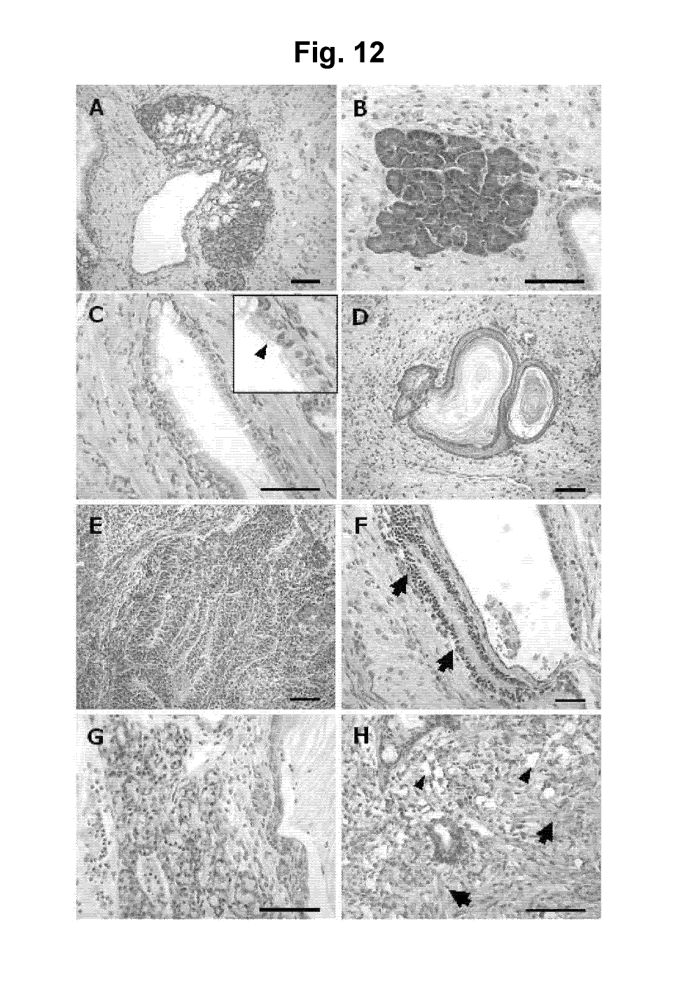

Transfer of the established ES cells into NOD-SCID mice resulted in the formation of teratomas containing a glandular stomach-like structure, exocrine pancreatic tissue, respiratory ciliary epithelium, keratinized and stratified squamous epithelium, neuroepithelial rosettes, pigmented retinal epithelium, sebaceous glands, adipocytes, and skeletal muscle bundles (FIG. 12). Karyotyping confirmed that the established cells possessed 40 chromosomes with XX.

Due to technical difficulties, we did not employ primordial or primary follicles, which are massively present in ovarian tissue, to establish ES cells. However, use of early secondary follicles was sufficient as a source of ES cells. Approximately 60% of the population of retrieved oocytes were at the early secondary follicle stage, and an average of more than 80 follicles were retrieved from one mouse. Considering average rates of maturation (50-60% in preliminary results; data not shown), cleavage (60%), blastocyst formation (80%), and ES cell establishment (20%) under optimal treatment conditions, at least five or six primary ES cell lines could be established from one animal. In fact, we succeeded in establishing ES cells from early secondary follicles in every animal that was euthanized.

Having described a preferred embodiment of the present invention, it is to be understood that variants and modifications thereof falling within the spirit of the invention may become apparent to those skilled in this art, and the scope of this invention is to be determined by appended claims and their equivalents.

### REFERENCES

1. Moore K L, Persaud T V N. Before We Are Born; Essentials of Embryology and Birth Defects. 5th ed. Philadelphia: W B Saunders, 1998.
2. Eppig J J, Schroeder A C. Capacity of mouse oocytes from preantral follicles to undergo embryogenesis and development to live young after growth, maturation, and fertilization in-vitro. Biol Reprod 1989; 41: 268-276.
3. Cortvrindt R, Smitz J, Van Steirteghem A C. In-vitro maturation, fertilization and embryo development of immature oocytes from early preantral follicles from prepuberal mice in a simplified culture system. Hum Reprod 1996; 11: 2656-2666.
4. Hubner K, Fuhrmann G, Christenson L K, Kehler J, Reinbold R, De La Fuente R, Wood J, Strauss J F 3^{rd}, Boiani M, Scholer H R. Derivation of oocytes from mouse embryonic stem cells. Science 2003; 300: 1251-1256.
5. Lenie S, Cortvrindt R, Adriaenssens T, Smitz J. A reproducible two-step culture system for isolated primary mouse ovarian follicles as single functional units. Bio Reprod 2004; 71: 1730-1738.
6. Stephanie A P, Hammad S, Lonnie D S, Teresa K W. Novel approach for the three-dimensional culture of granulose cell-oocyte complexes. Tissue Engineering 2003; 9: 1013-1021.
7. Roy S K, Greenwald G S. An enzymatic method for the dissociation of intact follicles from the hamster ovary: histological and quantitative aspect. Biol Reprod 1985; 32: 203-215.
8. Nayudu P L, Osborn S M. Factors influencing the rate of preantral and antral growth of mouse ovarian follicles in vitro. J Reprod Fertil 1992; 95: 349-362.
9. Roy S K, Greenwald G S. Methods of separation and in-vitro culture of pre-antral follicles from mammalian ovaries. Hum Reprod Update 1996; 2: 236-245.
10. Demeestere I, Delbaere A, Gervy C, Van den Bergh M, Devreker F, Englert Y. Effect of preantral follicle isolation technique on in-vitro follicular growth, oocyte maturation and embryo development in mice. Hum Reprod 2002; 17: 2152-2159.
11. Cortvrindt R G, Hu Y, Liu J, Smitz J E. Timed analysis of the nuclear maturation of oocytes in early preantral mouse follicle culture supplemented with recombinant gonadotropin. Fertil Steril 1998; 70: 1114-1125.
12. Smitz J E, Cortvrindt R G. The earliest stage of folliculogenesis in vitro. Reproduction 2002; 123: 185-202.
13. Cortvrindt R, Smitz J. Early preantral mouse follicle in vitro maturation: oocyte growth, meiotic maturation and granulose-cell proliferation. Theriogenology 1998; 49: 845-859.
14. Elvin J A, Yan C, Matzuk M M. Oocyte-expressed TGF-β superfamily members in female fertility. Mol Cell Endocrinol 2000; 159: 1-5
15. Dong J, Albertini D F, Nishimori K, Kumar T R, Lu N, Matzuk M M. Growth differentiation factor-9 is required during early ovarian folliculogenesis. Nature; 383: 531-535
16. Eppig J J, O'Brien M J, Pendola F L, Watanabe S. Factors affecting the developmental competence of mouse oocytes grown in vitro: follicle-stimulating hormones and insulin. Biol Reprod 1998; 59: 1445-1453
17. Cortvrindt, Hu Y, Smitz J. Recombinant luteinizing hormone as a survival and differential factor increases oocyte maturation in recombinant follicle stimulating hormone-supplemented mouse preantral follicle culture. Hum Reprod 1998; 13: 1292-1302
18. Roy S K, Treacy B J. Isolation and long-term culture of human preantral follicles. Fertil Steril 1993; 59: 783-790.
19. Epigg J J, O'Brien M J. Comparison of preimplantation developmental competence after mouse growth and development in vitro and in vivo. Theriogenology 1998; 49: 415-422
20. Liu J, Van Der Elst J, Van Den Broecke R, Dumortier F, Dhont M. Maturation of mouse primordial follicles by combination of grafting and in vitro culture. Bio Reprod 2000; 62: 1218-1223.
21. Eppig, J. J. and O'Brien, M. J., Development in vitro of mouse oocytes from primordial follicles. Biol. Reprod. 1996; 54, 197-207.
22. Carrell, D. T., Liu, L., Huang, I. and Peterson, C. M., Comparison of maturation, meiotic competence, and chromosome aneuploidy of oocytes derived from two protocols for in vitro culture of mouse secondary follicles. J. Assist. Reprod. Genet. 2005; 22, 347-354.
23. Mousset-Simeon, N., Jouannet, P., Le Cointre, L., Coussieu, C. and Poirot, C., Comparison of three in vitro culture systems for maturation of early preantral mouse ovarian follicles., Zygote 2005; 13, 167-175.
24. Cibelli, J. B., Grant, K. A., Chapman, K. B., Cunniff, K., Worst, T., Green, H. L., Walker, S. J., Gutin, P. H., Vilner, L., Tabar, V. et al., Parthenogenetic stem cells in nonhuman primates. Science 2002; 295, 819.
25. Gosden, R. G., Mullan, J., Picton, H. M., Yin, H. and Tan, S. L., Current perspective on primordial follicle cryopreservation and culture for reproductive medicine. Hum. Reprod. Update 2002; 8, 105-110.

1. A method for producing a preantral follicle-derived embryonic stem cell, which comprises the steps of:
(a) obtaining a preantral follicle from mammalian ovaries;
(b) growing the preantral follicle in vitro;
(c) maturing an oocyte in vitro present in the cultured preantral follicle;
(d) activating the matured oocyte for parthenogenesis;
(e) culturing the activated oocyte to form a blastocyst; and
(f) culturing inner cell mass (ICM) cells of the blastocyst to produce the preantral follicle-derived embryonic stem cell.

2. The method according to claim 1, wherein the preantral follicle is obtained by a mechanical method.

3. The method according to claim 1, wherein the preantral follicle is an early secondary follicle.

4. The method according to claim 1, wherein the mammal is human, bovine, sheep, ovine, pig, horse, rabbit, goat, mouse, hamster or rat.

5. The method according to claim 1, wherein the preantral follicle is grown in vitro to pseudoantral stage in step (b).

6. The method according to claim 1, wherein the growing the preantral follicle of step (b) is carried out in vitro by a single cell culture method.

7. The method according to claim 1, wherein the culturing of the activated oocyte of step (e) is carried out by a single cell culture method.

8. A preantral follicle-derived embryonic stem cell, wherein the embryonic stem cell has the same karyotype as an oocyte present in the preantral follicle, is stainable with alkaline phosphatase and capable of forming an embryonic body and teratoma.

9. The preantral follicle-derived embryonic stem cell according to claim 8, wherein the embryonic stem cell is derived from an early secondary follicle.

10. (canceled)

11. A preantral follicle-derived embryonic stem cell, wherein the embryonic stem cell has the same karyotype as an oocyte present in the preantral follicle, is stainable with alkaline phosphatase and capable of forming an embryonic body and teratoma, wherein the embryonic stem cell is prepared by the method of claim 1.
